SEQUENCE LISTING (30 sequences)

SEQ ID NO: 1 - protein, 829 aa, Homo sapiens [sequence omitted]

SEQ ID NO: 2 - DNA, 3171 bp, Homo sapiens
gcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacagatgaggatgatgccatctacacctacaatggggtggttgcttactccatccatagccaagaaccaaaggacccacacgacctcatgttcacaattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaacgaccatggcccagtccctgagccccgtcagatcaccatctgcaaccaaagccctgtgcgccacgtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaaaggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctaggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaa

SEQ ID NO: 3 - DNA, 160 bp, Artificial Sequence
  Primer
gcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctc

SEQ ID NO: 4 - DNA, 25 bp, Artificial Sequence
  Primer
acgtgcacctttctctgtctgacca

SEQ ID NO: 5 - DNA, 22 bp, Artificial Sequence
  Primer
aaaagcagaccagcaggaggaa

SEQ ID NO: 6 - DNA, 24 bp, Artificial Sequence
  Primer
cgggaaatcgtgcgtgacattaag

SEQ ID NO: 7 - DNA, 24 bp, Artificial Sequence
  Primer
tgatctccttctgcatcctgtcgg

SEQ ID NO: 8 - DNA, 25 bp, Artificial Sequence
  Antisense
agggttgagcattttacggcaggtg

SEQ ID NO: 9 - DNA, 25 bp, Artificial Sequence
  Antisense reverse control
atggacggcattttacgagttggga

SEQ ID NO: 10 - DNA, 25 bp, Artificial Sequence
  Antisense
catgggtgtcgggatgatggttggt

SEQ ID NO: 11 - DNA, 25 bp, Artificial Sequence
  Antisense reverse control
tggttggtagtagggctgtgggtac

SEQ ID NO: 12 - DNA, 25 bp, Artificial Sequence
  Antisense
acttgggcttgtggtcattctggtc

SEQ ID NO: 13 - DNA, 25 bp, Artificial Sequence
  Antisense reverse control
ctggtcttactggtgttcgggttca

SEQ ID NO: 14 - DNA, 25 bp, Artificial Sequence
  Antisense
cacaaactgctcatcctcacggtcg

SEQ ID NO: 15 - DNA, 25 bp, Artificial Sequence
  Antisense reverse control
gctggcactcctactcgtcaaacac

SEQ ID NO: 16 - DNA, 24 bp, Artificial Sequence
  Antisense
aatcttggtcttggtcggaggcgg

SEQ ID NO: 17 - DNA, 24 bp, Artificial Sequence
  Antisense reverse control
ggcggaggctggttctggttctaa

SEQ ID NO: 18 - DNA, 42 bp, Artificial Sequence
  Primer
ttcttcggatccgattgggtggttgctccaatatctgtccct

SEQ ID NO: 19 - DNA, 42 bp, Artificial Sequence
  Primer
ctgtacatgccaccgcccctcctgctgatccttaagtatgtg

SEQ ID NO: 20 - DNA, 63 bp, Artificial Sequence
  Primer
gggacaagtttgtacaaaaaagcaggctcagattgggtggttgctccaatatctgtccctgaa

SEQ ID NO: 21 - DNA, 84 bp, Artificial Sequence
  Primer
gtacagctttggacgggacctgggacctttgtagtggtagtggtagtgagatctactgggtcgaaagaacatgtttcaccaggg

SEQ ID NO: 22 - DNA, 38 bp, Artificial Sequence
  Primer
ctctgaccatgggacagtggaactcccgaagacacaag

SEQ ID NO: 23 - DNA, 32 bp, Artificial Sequence
  Primer
cagagacccatatgtaaattcagctgagtctc

SEQ ID NO: 24 - DNA, 58 bp, Artificial Sequence
  Primer
gggacaagtttgtacaaaaaagcaggctcgtgccgggcggtcttcagggaggctgaag

SEQ ID NO: 25 - DNA, 63 bp, Artificial Sequence
  Primer
gggacaagtttgtacaaaaaagcaggctcaatggactgggtcatccctccaatcaacttgcca

SEQ ID NO: 26 - DNA, 88 bp, Artificial Sequence
  Primer
ctgaggttgcccctgacgtgtctacacctgtccgtagtggtagtggtagtgagatctacttgggtcgaaagaacatgtttcaccaggg

SEQ ID NO: 27 - DNA, 60 bp, Artificial Sequence
  Primer
gggacaagtttgtacaaaaaagcaggctcagaagatttggactgcactcctggatttcag

SEQ ID NO: 28 - DNA, 88 bp, Artificial Sequence
  Primer
acgtccttaaggtttcacctgacgttgcgccgcgtagtggtagtggtagtgagatctacttgggtcgaaagaacatgtttcaccaggg

SEQ ID NO: 29 - DNA, 66 bp, Artificial Sequence
  Primer
gggacaagtttgtacaaaaaagcaggctcaatggactgggttattcctcccatcagctgcccagaa

SEQ ID NO: 30 - DNA, 88 bp, Artificial Sequence; identical to SEQ ID NO: 28
  Primer

(19) United States
(12) Patent Application Publication
(10) Pub. No.: US 2003/0194406 A1
(43) Pub. Date: Oct. 16, 2003

(54) P-cadherin as a target for anti-cancer therapy

(75) Inventors: Christoph Reinhard, Alameda, CA (US); Julie Klinger, Kensington, CA (US); Ann Jefferson, Oakland, CA (US); Jaime Escobedo, Alamo, CA (US); Fillipo Randazzo, Emeryville, CA (US); Jill Winter, Richmond, CA (US); Robert Goodson, Richmond, CA (US); Weimin Qi, Moraga, CA (US)

Correspondence Address: PILLSBURY WINTHROP, LLP, P.O. BOX 10500, MCLEAN, VA, 22102, US

(73) Assignee: CHIRON Corporation, Emeryville, CA

(21) Appl. No.: 10/158,123

(22) Filed: May 31, 2002

Related U.S. Application Data
(60) Provisional application No. 60/294,225, filed on May 31, 2001.

(51) Int. Cl. A61K 39/395; A61K 31/00

(52) U.S. Cl. 424/155.1; 514/1

(57) ABSTRACT

Method of treating or diagnosing cancers involving P-cadherin expression are provided using ligands that target P-cadherin, especially human anti-P-cadherin antibodies.

Also provided are screens for identifying anti-P-cadherin antibodies having therapeutic activity.

### CROSS REFERENCE TO RELATED APPLICATIONS

This application relates to U.S. Provisional Serial No. 60/208,871, filed on Jun. 2, 2000, and which is incorporated in its entirety by reference herein. This application claims priority to U.S. Provisional Serial No. 60/294,225 filed May 31, 2001 which is incorporated by reference in its entirety herein.

### BACKGROUND OF THE INVENTION

1. Field of the Invention

The invention relates to the use of P-cadherin as a target for treatment, prophylaxis and/or detection of cancers characterized by P-cadherin overexpression or upregulation.

2. Description of Related Art

The cadherin family of transmembrane glycoproteins play an important role in cell differentiation, cell migration, and intercellular adhesion. Family members include cadherins E-, P- and N-, all of which have cytoplasmic domains capable of interacting with beta and gamma catenins. In turn, beta and gamma catenins bind alpha-catenin, enabling cadherin-catenin structures to complex with cytoskeletal actin.

Cadherins have been linked to various types of neoplastic conditions. For example, expression of P-cadherin, a calcium-dependent cellular adhesion protein, has been reported in poorly differentiated and invasive bladder carcinoma cells. Such bladder carcinoma cells exhibit reduced E-cadherin expression. (Mialhe, A. et al., J. Urol. 164:826 (2000)). Down-regulation of E-cadherin and P-cadherin has also been associated with cultured neoplastic prostate cells. (Wang, J. et al., Urol. Res. 5:308 (2000)). The development of human colorectal cancer has been attributed, at least in part, to a decrease in cellular levels of the E-cadherin/catenin complex. (Debruyne, P. et al., Acta Gastroenterol. Belg. 62(4):393 (1999)). Aberrant up-regulation of P-cadherin was recently reported to be associated with proliferative cell phenotypes that may be related to neoplastic transformation of tissues of the gastrointestinal tract, particularly metaplastic and adenomatous polyps. (Sanders, D. S., et al., J. Pathol. 190(5):526 (2000)). However, the direct correlation that P-cadherin is overexpressed in many colon cancers was not previously known.

One report from Jankowski's group observed that P-Cadherin was aberrantly expressed from the earliest morphologically identifiable stage of colonocyte transformation, prior to changes in E-Cadherin, catenin, and APC expression/mutation. But the P-Cadherin expression alone did not predict tissue morphology, and such expression was independent of that of associated cadherin and catenin (Hardy et al., Gut (4):513-519 (2002)). Another report from Hardisson's group observed that the P-Cadherin-positive tumors were negative for estrogen and progesterone receptors, whereas E-Cadherin expression is associated with positive estrogen and progesterone receptors, indicating that a P-Cadherin antibody can be used for treatment of the ER negative cancer population (Gammallo et al., Mod Pathol 14(7):650-654 (2001)).

### SUMMARY OF THE INVENTION

The present invention relates to the use of P-cadherin, as a target for cancer diagnosis, prophylaxis or therapy. P-cadherin is a transmembrane protein which is part of a class of proteins which link to the cellular cytoskeleton through the formation of complexes by intimate transmembrane binding with cytosolic proteins, the catenins. Cadherins are thought to be the key players in epithelial cell-cell adhesion. Other major roles include the determination of cell phenotypes and involvement in cell dynamics, including migration and the dissemination of tumor cells. It is known that P-cadherin (placental cadherin) is commonly expressed in epithelial tissues. The relative role and differential action of P-cadherin has not been previously described.

With respect thereto, the present invention hinges on the discovery that certain cancer types, particularly some digestive cancer types, e.g., colon cancer, are characterized by the upregulation and the overexpression of P-cadherin relative to normal cells and that moieties which bind and/or inhibit P-cadherin expression and/or activity may be used to treat or prevent cancers characterized by P-cadherin overexpression such as digestive cancers including colon cancer.

Specifically, microchip array data from multiple human colon cancer samples demonstrate the up-regulation of the P-cadherin transcript in these samples. In situ hybridization data from these tissue samples localized the transcript in colon cancer cells. P-cadherin was upregulated in more than 5 fold in 50% of tumor samples obtained from 33 colon cancer patients, as analyzed via microarray chips. This cell surface adhesion protein, therefore, appears to be aberrantly expressed in a substantial number of the colon cancer tumors sampled.

Immunohistochemical analysis of human colon cancer samples, using P-cadherin specific monoclonal antibodies, indicate more P-cadherin gene product is present in cancerous tissue of the colon than in normal human colon tissue samples. Using a commercially available mouse monoclonal antibody raised against P-cadherin, cell adhesion and cell proliferation were blocked in a culture comprising P-cadherin expressing cell lines. Tissue distribution of P-cadherin in normal and cancerous tissues was also assessed using commercially available anti-P-cadherin monoclonal antibodies. Expression of P-cadherin in colon cancer cells was further confirmed via immunohistochemistry. Immunochemistry results also indicate that some normal layers of epithelium, such as oral cavity and vaginal epithelium, express P-cadherin. Expression of P-cadherin was also observed in normal pancreas and adrenal gland samples as well. Human colon cancer cell explants (KM12 cells) grown in nude mice were found to express P-cadherin. P-cadherin expression has been detected immunohistochemically in other cancers, particularly lung cancer, stomach cancer and breast cancer.

Using antisense technology, knock out studies have indicated that P-cadherin is important in the regulation of cancer cell proliferation. In particular, antisense data obtained using a cell line that expressed moderate levels of P-cadherin indicate that inhibition of expression negatively affects cell proliferation.

Based on these observations, i.e., that P-cadherin is overexpressed in some human cancer types, e.g., human colon cancer, and that inhibiting and/or blocking P-cadherin expression or function may cumulate to cancer cell proliferation, migration and/or proliferation, the present invention is directed toward a method of diagnosing, preventing or treating cancers characterized by the overexpression and/or upregulation of P-cadherin by targeting or detecting P-cadherin.

In the case of prevention or treatment of cancers involving P-cadherin overexpression or upregulation, the preset invention generally will involve the administration of at least one “P-cadherin antagonist”. By contrast, in the case of diagnosis of cancers characterized by P-cadherin overexpression, the present invention will involve determining levels of expression of P-cadherin in a tissue or cell sample relative to a control (normal) tissue or cell sample, and correlating levels of expression to a positive or negative diagnosis of a cancer characterized by P-cadherin upregulation, e.g. colon cancer.

The present invention also relates to the production of specific “P-cadherin antagonists” (defined infra), particularly antibodies or antibody fragments, or small molecules that specifically bind P-cadherin, as well as ribozymes and antisense oligonucleotides that modulate P-cadherin expression.

Additionally, the present invention relates to novel pharmaceutical compositions containing such antagonists.

### BRIEF DESCRIPTION OF THE FIGURES

The invention is illustrated in the following Figures in which:

FIG. 1 shows P-cadherin expression on the surface of A-431 cells and SW620 cells measured using two anti-P-cadherin antibodies (NCC-CAD-299, obtained from Zymed and RDI-PCADHER abm, an anti-P-cadherin antibody obtained from RDI) and an irrelevant (control) isotype matched IgG1 antibody.

FIG. 2 shows the effect of two anti-P-cadherin antibodies (NCC-CAD-299, obtained from Zymed and RDI-CADHER abm, an anti-P-cadherin antibody obtained from RDI), as well as an irrelevant isotype matched IgG1 on cell-cell adhesion in A-431 cell cultures.

FIG. 3 shows the effect of two anti-P-cadherin antibodies (NCC-CAD-299, obtained from Zymed and RDI-PCADHER abm, an anti-P-cadherin antibody obtained from RDI), as well as an irrelevant isotype matched IgG1 on the proliferation of A431 and SW620 cell cultures.

FIGS. 4 and 5 contain the results of IHC analysis relating to the expression of P-cadherin and E-cadherin on different epithelium.

FIG. 6 contains the results of IHC analysis relating to the expression of P-cadherin on various normal tissues.

FIG. 7 contains the results of IHC analysis relating to the expression of P-cadherin on some normal breast and cancerous breast cancers.

FIG. 8 contains the results of IHC analysis relating to the P-cadherin expression on normal colon, tumor colon and metastatic colon.

FIG. 9 contains the results of IHC analysis that detected P-cadherin in a human colon cancer model (KM12 human colon cancer in a nude mouse).

FIG. 10 shows the expression of P-cadherin in a baculovirus system.

### DETAILED DESCRIPTION OF THE INVENTION

In a related application, U.S. Provisional Serial No. 60/208,871, filed Jun. 2, 2000, incorporated by reference herein, the present Assignee disclosed a number of polynucleotide sequences that are differentially expressed in colon cancer cells. These polynucleotide sequences included P-cadherin. The present invention relates to the use of P-cadherin as a target for therapeutic intervention in the treatment and/or prophylaxis of cancers, like colon cancer, that are characterized by tumor tissues that express P-cadherin on their surface, preferably at higher levels than normal tissues. Also, the invention relates to the use of P-cadherin as a diagnostic target.

Various data and observations suggest that P-cadherin is an appropriate target for cancer therapy. For example, chip microarray data indicates that P-cadherin is upregulated more than five-fold in approximately 50% of colon cancer patients which have been analyzed. This microarray data for 33 patients is summarized in Table 1 (infra) and suggests that the overexpression of P-cadherin correlates to the presence of some cancers. Moreover, as P-cadherin is a member of a large family of proteins including cadherins and cadherin-like proteins that are believed to be instrumental in cell-cell adhesion, it was theorized that this protein may play a causal role in P-cadherin associated cancers, perhaps by facilitating metastasis.

Secondly, it has been shown that antisense oligonucleotides which are complementary to the P-cadherin transcript when transfected into cancer cells that express moderate levels of P-cadherin, inhibit the proliferation thereof. This data is further suggestive of the efficacy of P-cadherin as an appropriate therapeutic target as it indicates that P-cadherin expression may affect (promote) cancer cell proliferation (These antisense experimental results are discussed in further detail in the experimental example section).

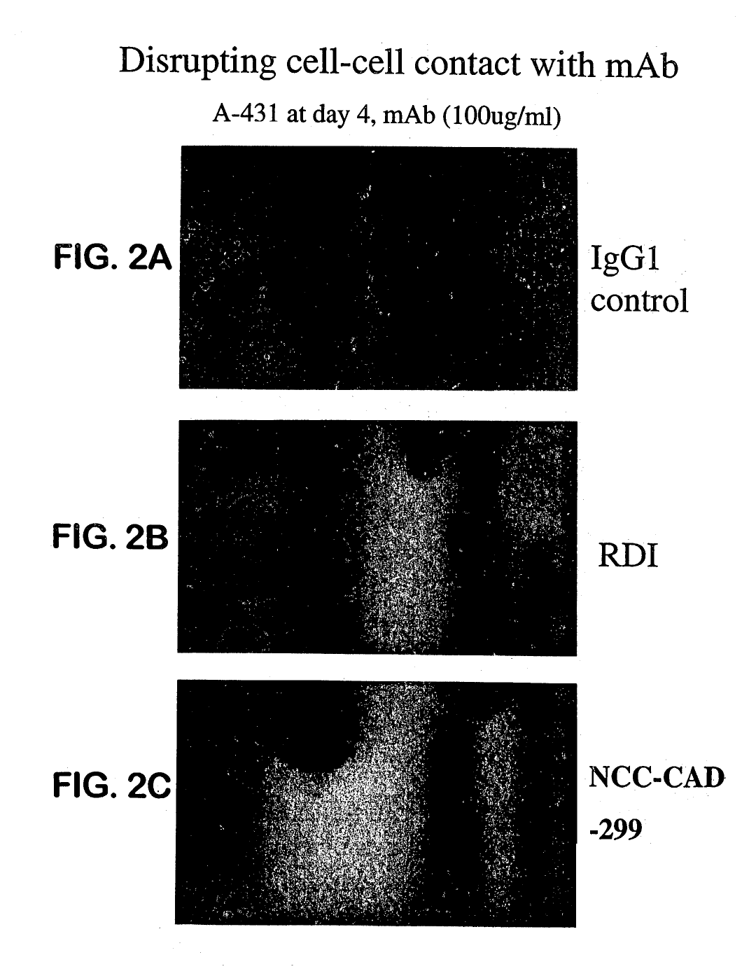

Thirdly, it has been shown that a monoclonal antibody specific to P-cadherin block the adhesion of P-cadherin expressing cells in tissue culture. As discussed in further detail in the examples, it has been shown that NCC-CAD-299, a mouse IgG1 monoclonal antibody obtained from Zymed, which binds human P-cadherin, blocks the adhesion of A-431, an epithelial tumor cell line which expresses moderate levels of P-cadherin. By contrast, another tested mouse IgG1 anti-human P-cadherin monoclonal antibody, RDI-PCADHER abm available from Research Diagnostics, Inc. (RDI), and an irrelevant mouse IgG1 antibody did not affect A-431 cell adhesion. These results are contained in FIG. 2. (With respect to the anti-P-cadherin antibody that did not block cell adhesion, it is theorized that this antibody may bind to a distinct epitope not involved in cell adhesion, or may be attributable to different activities or affinities of these two antibodies). These results in combination substantiate that ligands specifically bind P-cadherin, e.g. antibodies or antibody fragments or which inhibit P-cadherin expression, e.g. antisense oligos or ribozymes, may be useful for the treatment of P-cadherin associated cancers. Particularly, these results suggest that the anti-P-cadherin ligands may inhibit metastasis by preventing or inhibiting P-cadherin expressing cancer cells from migrating, adhering and producing a tumor at different sites.

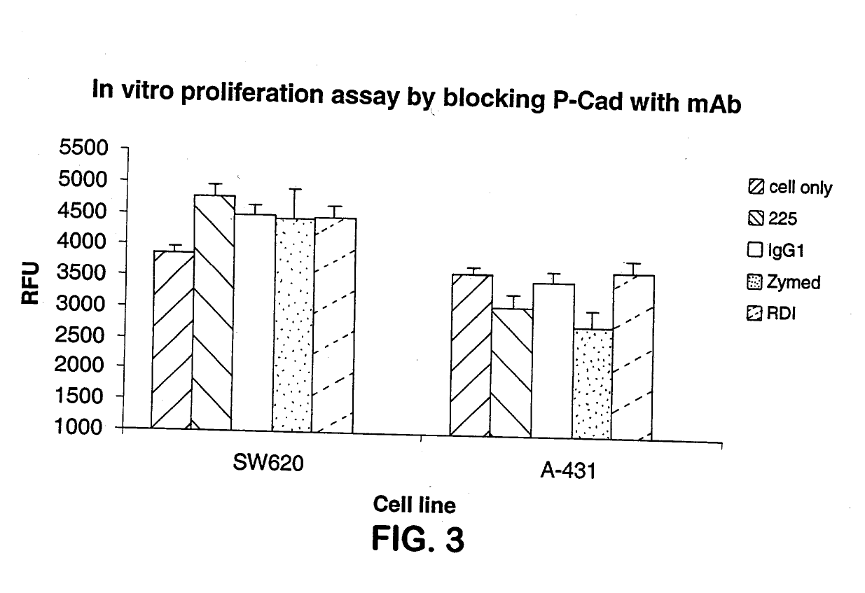

Fourthly, it has been shown that monoclonal antibodies specific to P-cadherin inhibit the proliferation of P-cadherin expressing cell lines in tissue culture. As discussed in further detail in the examples, it has been shown (results in FIG. 3) that a commercially available monoclonal antibody NCC-CAD-299 (obtained from Zymed) inhibited the growth of A-431 cells in tissue culture. By contrast, an irrelevant mouse IgG1 monoclonal antibody did not inhibit cell proliferation. Neither did the other tested anti-P-cadherin monoclonal antibody, RDI-PCADHER abm obtained from RDI (The differences in functional behavior of the two tested anti-P-cadherin monoclonal antibodies is again theorized to be potentially attributable to epitopic, avidity and/or affinity differences between these two antibodies).

These results provide evidence that disrupting or blocking P-cadherin in cancer cells by the administration of a P-cadherin antagonist will inhibit the growth of cancer cells that overexpress P-cadherin or will inhibit the initiation of P-cadherin associated cancers.

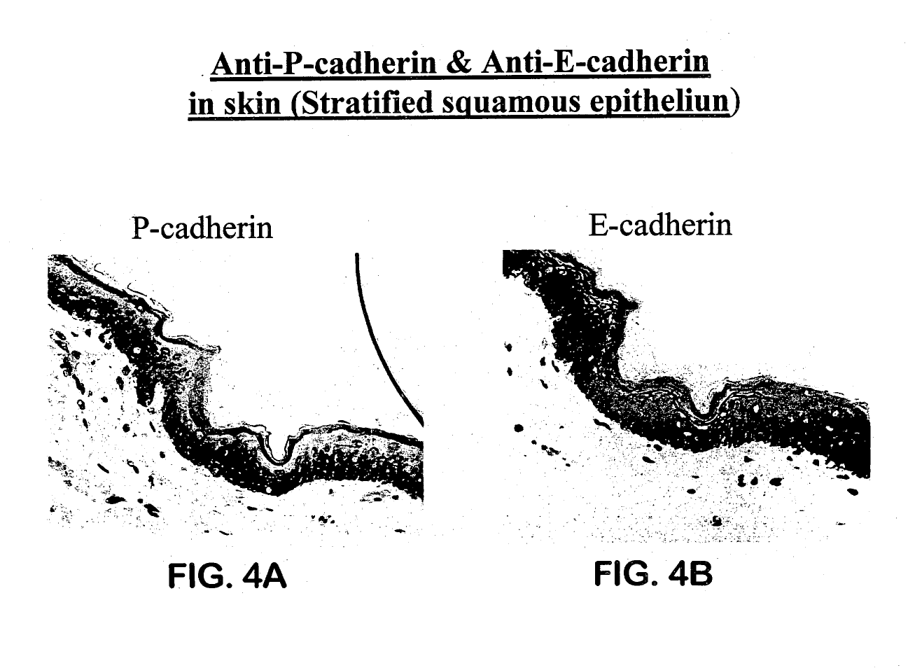

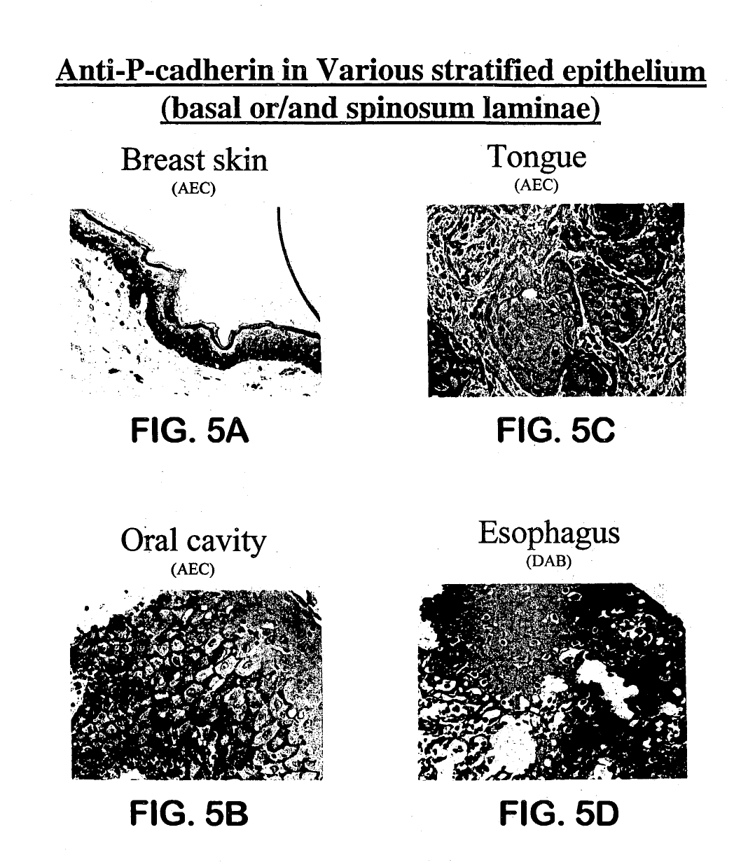

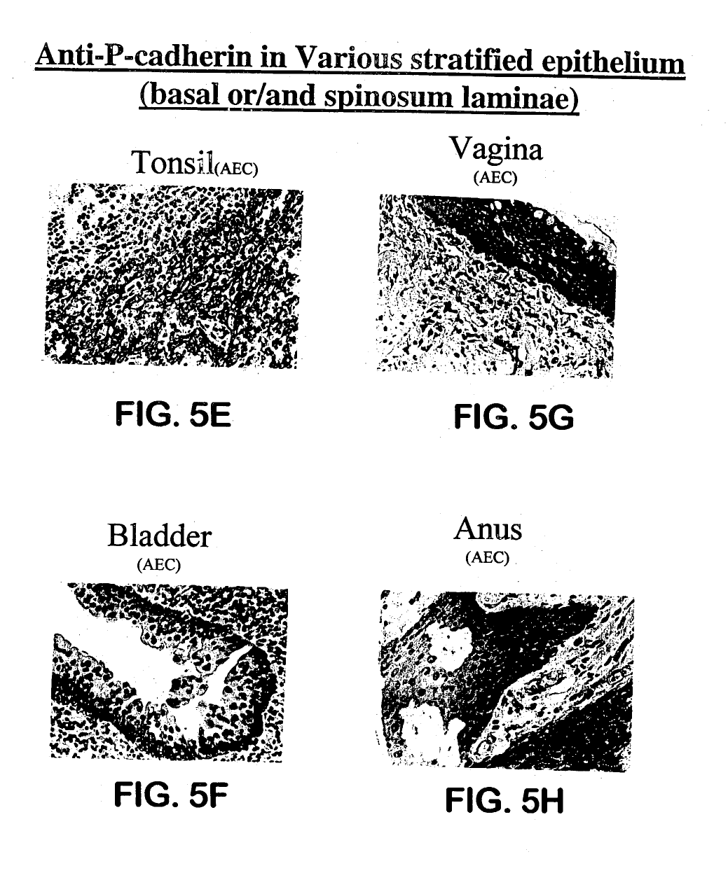

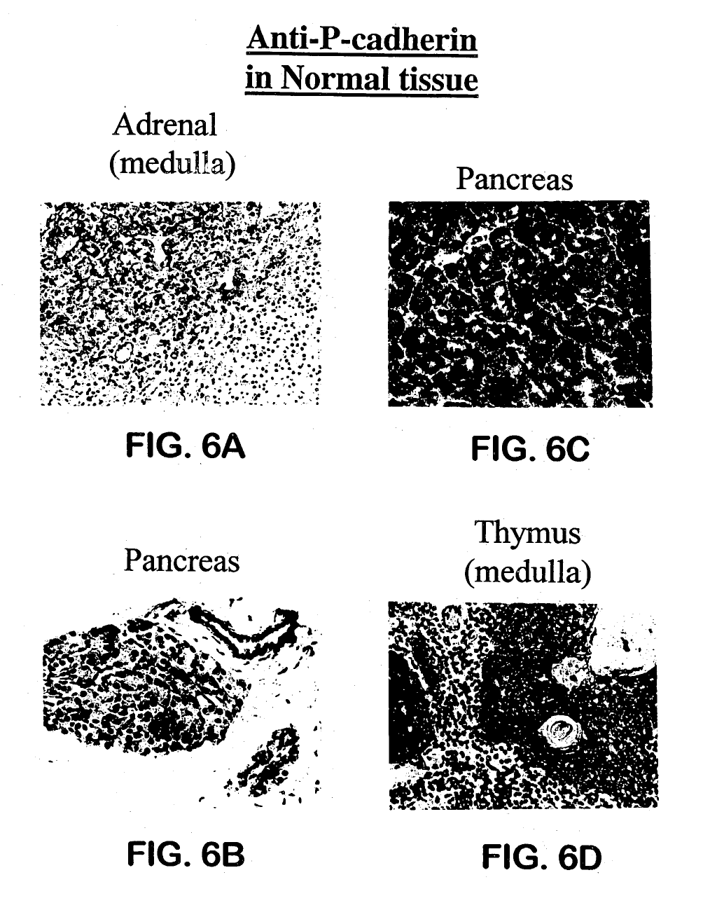

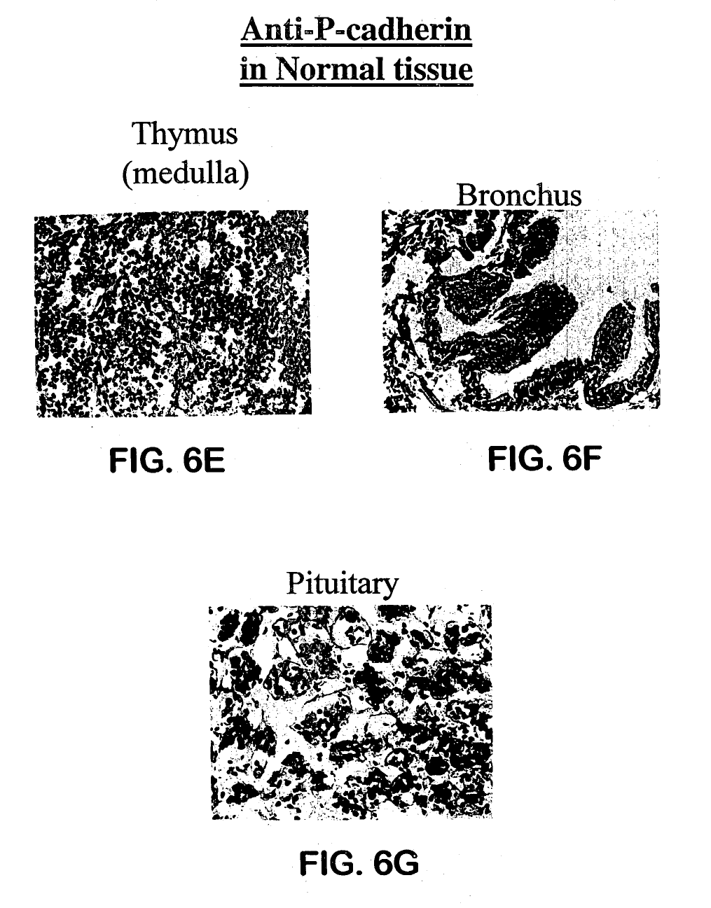

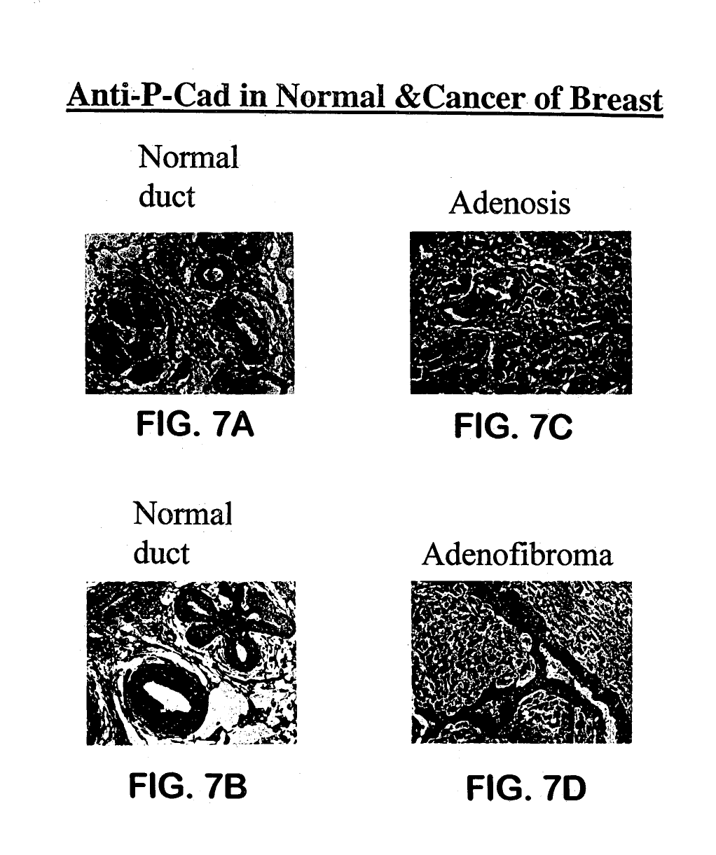

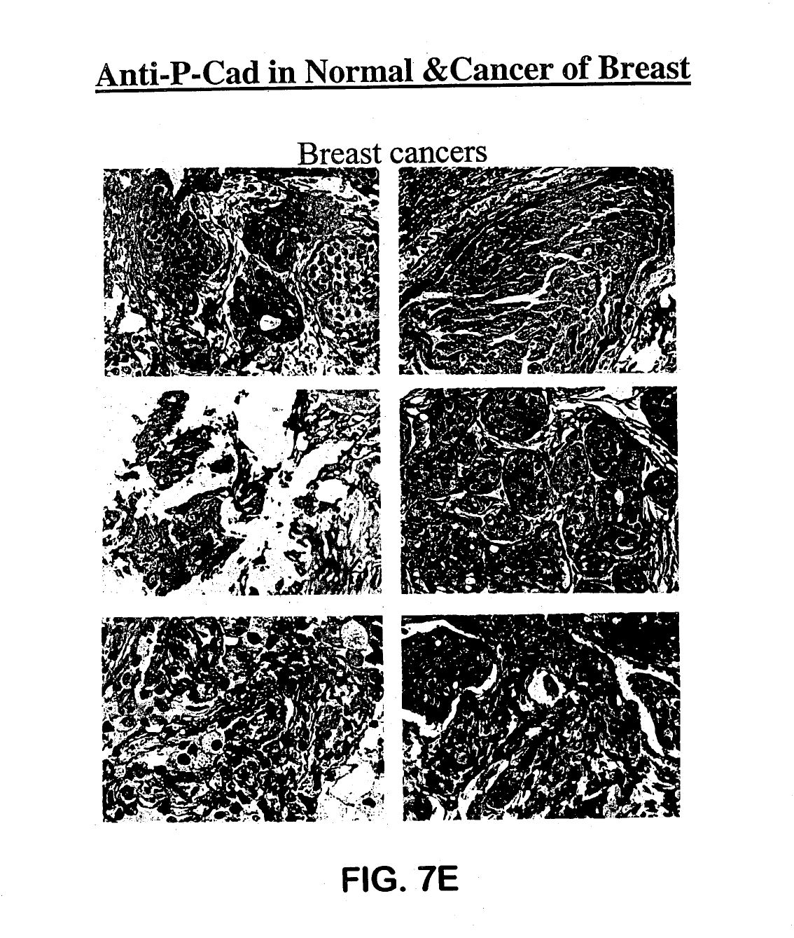

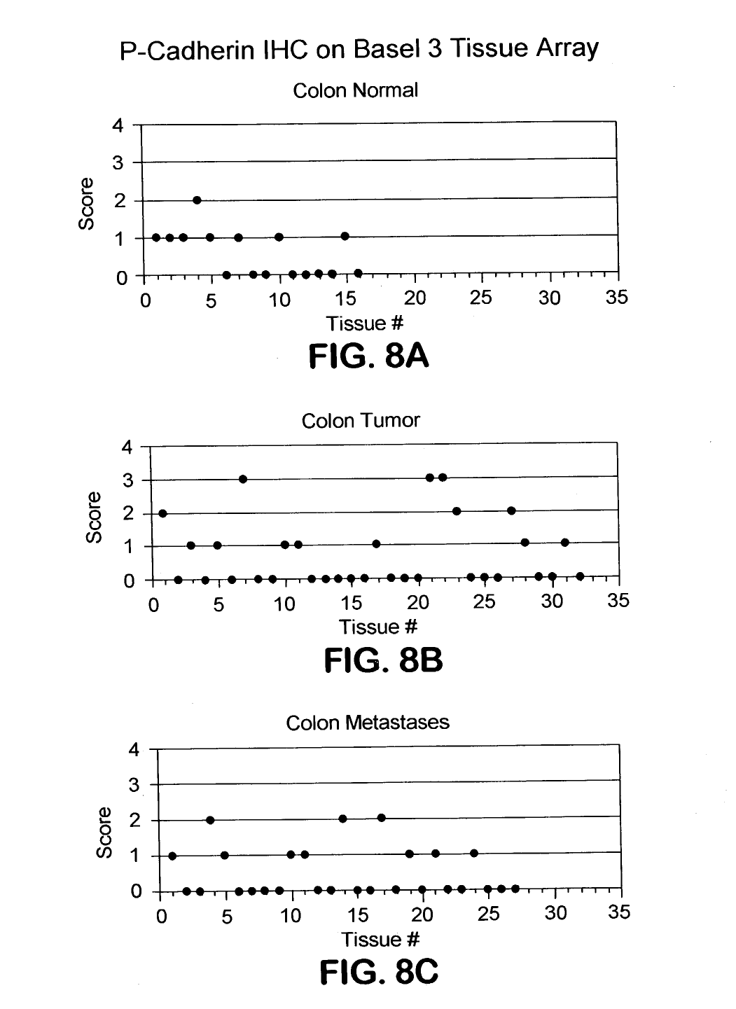

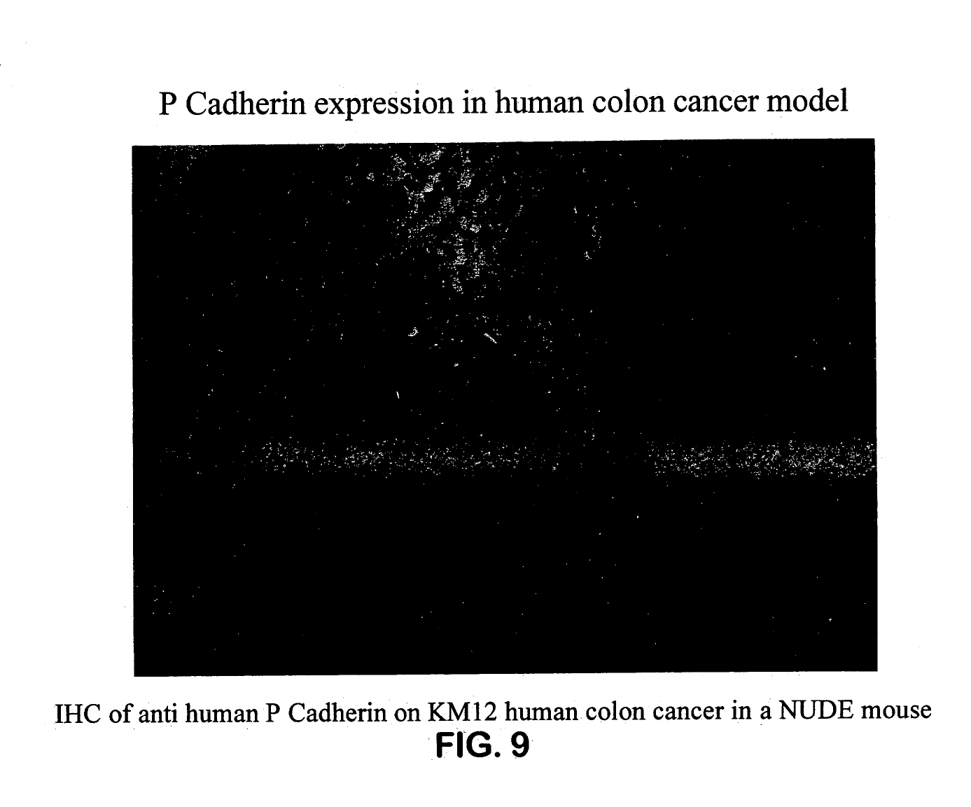

Fifthly, it has been shown using commercially available antibodies which specifically bind P-cadherin to evaluate P-cadherin tissue distribution immunohistochemically (IHC) suggests that P-cadherin is expressed at higher levels in some cancers, especially some digestive cancers. Similar staining results have been obtained using two different anti-P-cadherin monoclonal antibodies. Some of the IHC results are contained in FIGS. 4-10. FIGS. 4 and 5 contain IHC results evaluating P-cadherin expression in different types of epithelial tissue, i.e., skin, breast, esophagus, bladder, vagina, tongue, tonsil and anus. FIG. 6 contains IHC data which suggests that P-cadherin may be expressed on some normal tissues, particularly adrenal, thymus, pancreas, bronchus and pituitary. FIG. 7 contains IHC data suggesting that P-cadherin is expressed on normal tissues and at higher levels on cancerous breast tissue. FIG. 8 contains results of a 3 tissue array (colon normal, colon tumor, and colon metastasis) which determined by IHC P-cadherin expression on these different tissues. The results indicate that P-cadherin is expressed at higher levels in the cancerous tissues than in normal colon tissues. FIG. 9 show P-cadherin expression in a human colon cancer model (KM12 human cancer in nude mice). These IHC results suggests that this or other human colon cancer models will be useful for confirming the in vivo efficacy of potential P-cadherin antagonists, e.g. anti-P-cadherin monoclonal antibodies, or antisense oligonucleotides.

Finally, P-cadherin has been used to pan human scFV antibody libraries. While these results are not reported herein, a number of possible P-cadherin binding scFv sequences have been identified using known methods.

These results cumulatively provide compelling evidence that the administration of P-cadherin antagonists should provide an efficious means of treating cancers characterized by P-cadherin overexpression, as these antagonists may inhibit the proliferation, migration and/or adhesion of P-cadherin expressing cancer cells. This should result in reduced tumor growth and potentially inhibit tumor metastasis.

Also, as P-cadherin expression is elevated in certain cancers, and apparently correlates to cancer cell proliferation, these results suggest that assays which measure the levels of P-cadherin expression in cells may be used to determine the proliferative potential of such cells.

Thus, based on the foregoing, the invention provides novel methods of diagnosing, treating and/or preventing cancers characterized by P-cadherin overexpression based on the detection of P-cadherin or the use of P-cadherin as a target for therapeutic intervention or prophylactic intervention.

It should be understood, however, that this invention is not limited to the particular embodiments described, as such may, of course, vary. It is also to be understood that the terminology used herein is for the purpose of describing particular embodiments only, and is not intended to be limiting.

Unless defined otherwise, all technical and scientific terms have the same meaning as commonly understood by one of ordinary skill in the art to which this invention belongs. Although any methods and materials similar or equivalent to those described herein can be used in the practice or testing of the present invention, the preferred methods and materials are now described. All publications and patent applications mentioned herein are incorporated by reference to disclose and describe the methods and/or materials in connection with which the publications are cited.

It is also noted that herein the singular forms, “a”, “and” and “the” include plural references unless the context clearly dictates otherwise. Thus, for example, reference to “an anti-P-cadherin antagonist” includes a plurality of such antagonists, and reference to “a P-cadherin expressing cancer cell” includes reference to one or more such cells and equivalents thereof known to those skilled in the art.

The publications and applications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In order to further describe the invention, the following specific and more general terms are defined as follows.

### Definitions

“P-cadherin antagonist” is a compound that specifically binds to P-cadherin protein, preferably human P-cadherin or binds to a P-cadherin polynucleotide or fragment thereof, and/or a compound that inhibits the activity and/or expression of P-cadherin protein or polynucleotide. Examples thereof include ligands that bind P-cadherin such as antibodies and antibody fragments, recombinant or native, other binding partners such as receptors, synthetic peptides (e.g. expressed by peptide libraries) and non-proteinaceous binding partners such as small molecules. Other antagonists include ribozymes or antisense oligonucleotides and/or compounds that modify P-cadherin gene structure so as to inhibit P-cadherin expression.

Preferably P-cadherin antagonists, e.g., anti-P-cadherin antibodies will bind P-cadherin with greater affinity than other cadherins, e.g., E-cadherin, N-cadherin and H-cadherin. More preferably, the relative binding affinity of P-cadherin to E-cadherin or another non-P-cadherin will be at least 5/1, more preferably at least 10/1, 20/1, 50/1, 100/1 or 1000/1 or the antibody will not detectably bind to cadherins other than P-cadherin. Also, preferably P-cadherin antibodies will be selected that bind to critical regions of the protein, such as the EC1 binding region.

P-cadherin antagonists specifically includes molecules that bind to specific portions of the P-cadherin protein, as described in great detail infra, e.g., those that bind the EC1 domain, those that inhibit strand dimer formula, those that inhibit cis dimer formula, those that interfere with calcium binding, and those that interfere with protein confirmation, e.g., those that permit alignment of specific domains. This is disclosed in greater detail infra, by reference to publication that described in detail that P-cadherin structure and function of specific portions of the protein.

“P-cadherin polypeptide” refers to placental cadherin, a polypeptide that is in the cadherin family of proteins. This family includes protein members that are involved in cell-cell adhesion. P-cadherin polypeptides are intended to include P-cadherins of different species, e.g., human, murine or another species that naturally expresses the polypeptide, as well as portions of fragments thereof. Such polypeptides further include allelic variants of P-cadherin where such variants can be of the same or different species origin. In general, variant polypeptides have a sequence that is at least 80%, usually at least 90%, and more usually at least about 98%, sequence identity with a P-cadherin polypeptide disclosed herein, as measured by BLAST 2.0 using the parameters described above.

Preferably, P-cadherin will refer to human P-cadherin which is an 882 amino acid protein having a putative signal peptide, putative precursor region, an extracellular domain containing internal repeats and a highly hydrophobic transmembrane region. Preferably, P-cadherin will be human cadherin, a homolog or a fragment thereof. The amino acid sequence of human P-cadherin is set forth below (SEQ ID NO: 1):

MGLPRGPLASLLLLQVCWLQCAASEPCRAVFREAEVTLEAGGAEQEPGQA
LGKVFMGCPGQEPALFSTDNDDFTVRNGETVQERRSLKERNPLKIFPSKR
ILRRHKRDWVVAPISVPENGKGPFPQRLNQLKSNKDRDTKIFYSITGPGA
DSPPEGVFAVEKETGWLLLNKPLDREETAKYELFGHAVSENGASVEDPMN
ISIIVTDQNDHKPKFTQDTFRGSVLEGVLPGTSVMQVTATDEDDAIYTYN
GVVAYSIHSQEPKDPHDLMFTIHRSTGTISVISSGLDREKVPEYTLTIQA
TDMDGDGSTTTAVAVVEILDANDNAPMFDPQKYEAHVPENAVGHEVQRLT
VTDLDAPNSPAWRATYLIMGGDDGDHFTITTHPESNQGILTTRKGLDFEA
KNQHTLYVEVTNEAPFVLKLPTSTATIVVHVEDVNEAPVFVPPSKVVEVQ
EGIPTGEPVCVYTAEDPDKENQKISYRILRDPAGWLAMDPDSGQVTAVGT
LDREDEQFVRNNIYEVNVLAMDNGSPPTTGTGTLLLTLIDVNDHGPVPEP
RQITICNQSPVRHVLNTTDKDLSPHTSPEQAQLTDDSDIYWTAEVNEEGD
TVVLSLKKELKQDTYDVHLSLSDHGNKEQLTVIRATVCDCHGHVETCPGP
WKGGFILPVLGAVLALLFLLLVLLLLVRKKRKIKEPLLLPEDDTRDNVFY
YGEEGGGEEDQDYDITQLHRGLEARPEVVLRNDVAPTITPTPMYRPRPAN
PDETGNFIIENLKAANTDPTAPPYDTLLVFDYEGSGSDAASLSSLTSSAS
DQDQDYDYLNEWGSRFKKLADMYGGGEDD

[This sequence was reported in Shimoyoma et al., J. Cell Biol. 109: 1787-1794 (1989) incorporated by reference in its entirety herein.]

“P-cadherin polynucleotide,” “P-cadherin nucleic acid” and “P-cadherin DNA” are used interchangeably herein and refers to a polynucleotide that encodes P-cadherin polypeptide or a variant or fragment thereof. In particular, such variants include sequences that posses at least 95% sequence identity to a naturally occurring P-cadherin or a fragment thereof. Preferably, a P-cadherin polynucleotide will encode human P-cadherin or a fragment or variant thereof. The nucleic acid sequence for human P-cadherin was also reported by Shimoyoma et al. (Id.) and is set forth below (SEQ ID NO: 2):

ORIGIN

| | |
|---|---|
| 1 | gcggaacacc ggcccgccgt cgcggcagct gcttcacccc tctctctgca gccatggggc |
| 61 | tccctcgtgg acctctcgcg tctctcctcc ttctccaggt ttgctggctg cagtgcgcgg |
| 121 | cctccgagcc gtgccgggcg gtcttcaggg aggctgaagt gaccttggag gcgggaggcg |
| 181 | cggagcagga gcccggccag gcgctgggga aagtattcat gggctgccct gggcaagagc |
| 241 | cagctctgtt tagcactgat aatgatgact tcactgtgcg gaatggcgag acagtccagg |
| 301 | aaagaaggtc actgaaggaa aggaatccat tgaagatctt cccatccaaa cgtatcttac |
| 361 | gaagacacaa gagagattgg gtggttgctc caatatctgt ccctgaaaat ggcaagggtc |
| 421 | ccttccccca gagactgaat cagctcaagt ctaataaaga tagagacacc aagattttct |
| 481 | acagcatcac ggggccgggg gcagacagcc cccctgaggg tgtcttcgct gtagagaagg |
| 541 | agacaggctg gttgttgttg aataagccac tggaccggga ggagattgcc aagtatgagc |
| 601 | tctttggcca cgctgtgtca gagaatggtg cctcagtgga ggaccccatg aacatctcca |
| 661 | tcatcgtgac cgaccagaat gaccacaagc ccaagtttac ccaggacacc ttccgaggga |
| 721 | gtgtcttaga gggagtccta ccaggtactt ctgtgatgca ggtgacagcc acagatgagg |
| 781 | atgatgccat ctacacctac aatggggtgg ttgcttactc catccatagc caagaaccaa |
| 841 | aggacccaca cgacctcatg ttcacaattc accggagcac aggcaccatc agcgtcatct |
| 901 | ccagtggcct ggaccgggaa aaagtccctg agtacacact gaccatccag gccacagaca |
| 961 | tggatgggga cggctccacc accacggcag tggcagtagt ggagatcctt gatgccaatg |
| 1021 | acaatgctcc catgtttgac ccccagaagt acgaggccca tgtgcctgag aatgcagtgg |
| 1081 | gccatgaggt gcagaggctg acggtcactg atctggacgc ccccaactca ccagcgtggc |
| 1141 | gtgccaccta ccttatcatg ggcggtgacg acggggacca ttttaccatc accacccacc |
| 1201 | ctgagagcaa ccagggcatc ctgacaacca ggaagggttt ggattttgag gccaaaaacc |
| 1261 | agcacaccct gtacgttgaa gtgaccaacg aggccccttt tgtgctgaag ctcccaacct |
| 1321 | ccacagccac catagtggtc cacgtggagg atgtgaatga ggcacctgtg tttgtcccac |
| 1381 | cctccaaagt cgttgaggtc caggagggca tccccactgg ggagcctgtg tgtgtctaca |
| 1441 | ctgcagaaga ccctgacaag gagaatcaaa agatcagcta ccgcatcctg agagacecag |
| 1501 | cagggtggct agccatggac ccagacagtg ggcaggtcac agctgtgggc accctcgacc |
| 1561 | gtgaggatga gcagtttgtg aggaacaaca tctatgaagt catggtcttg gccatggaca |
| 1621 | atggaagccc tcccaccact ggcacgggaa cccttctgct aacactgatt gatgtcaacg |
| 1681 | accatggccc agtccctgag ccccgtcaga tcaccatctg caaccaaagc cctgtgcgcc |
| 1741 | acgtgctgaa catcacggac aaggacctgt ctccccacac ctcccctttc caggcccagc |
| 1801 | tcacagatga ctcagacatc tactggacgg cagaggtcaa cgaggaaggt gacacagtgg |
| 1861 | tcttgtccct gaagaagttc ctgaagcagg atacatatga cgtgcacctt tctctgtctg |
| 1921 | accatggcaa caaagagcag ctgacggtga tcagggccac tgtgtgcgac tgccatggcc |
| 1981 | atgtcgaaac ctgccctgga ccctggaaag gaggtttcat cctccctgtg ctgggggctg |
| 2041 | tcctggctct gctgttcctc ctgctggtgc tgcttttgtt ggtgagaaag aagcggaaga |
| 2101 | tcaaggagcc cctcctactc ccagaagatg acacccgtga caacgtcttc tactatggcg |
| 2161 | aagagggggg tggcgaagag gaccaggact atgacatcac ccagctccac cgaggtctgg |
| 2221 | aggccaggcc ggaggtggtt ctccgcaatg acgtggcacc aaccatcatc ccgacaccca |
| 2281 | tgtaccgtcc taggccagcc aacccagatg aaatcggcaa ctttataatt gagaacctga |
| 2341 | aggcggctaa cacagacccc acagccccgc cctacgacac cctcttggtg ttcgactatg |
| 2401 | agggcagcgg ctccgacgcc gcgtccctga gctccctcac ctcctccgcc tccgaccaag |
| 2461 | accaagatta cgattatctg aacgagtggg gcagccgctt caagaagctg gcagacatgt |
| 2521 | acggtggcgg ggaggacgac taggcggcct gcctgcaggg ctggggacca aacgtcaggc |
| 2581 | cacagagcat ctccaagggg tctcagttcc cccttcagct gaggacttcg gagcttgtca |
| 2641 | ggaagtggcc gtagcaactt ggcggagaca ggctatgagt ctgacgttag agtggttgct |
| 2701 | tccttagcct ttcaggatgg aggaatgtgg gcagtttgac ttcagcactg aaaacctctc |
| 2761 | cacctgggcc agggttgcct cagaggccaa gtttccagaa gcctcttacc tgccgtaaaa |
| 2821 | tgctcaaccc tgtgtcctgg gcctgggcct gctgtgactg acctacagtg gactttctct |
| 2881 | ctggaatgga accttcttag gcctcctggt gcaacttaat tttttttttt aatgctatct |
| 2941 | tcaaaacgtt agagaaagtt cttcaaaagt gcagcccaga gctgctgggc ccactggccg |
| 3001 | tcctgcattt ctggtttcca gaccccaatg cctcccattc ggatggatct ctgcgttttt |
| 3061 | atactgagtg tgcctaggtt gccccttatt ttttattttc cctgttgcgt tgctatagat |
| 3121 | gaagggtgag gacaatcgtg tatatgtact agaacttttt tattaaagaa a |

“Cells which express P-cadherin” refer to any cell which expresses detectable levels of P-cadherin. Detection can be determined by well known protein detection methods such as enzyme-linked immunosorbant assay (ELISA), radioimmunoassay (RIA), or immunoflourescence, or by detection of the transcript encoding P-cadherin such as by polymerase chain reaction, or in situ hybridization. Other methods for detecting specific polynucleotides or polypeptides are identified infra and are well known to those skilled in the art.

“Cells which overexpress or upregulate P-cadherin” refer to cells wherein the P-cadherin protein or transcript is expressed at higher levels than in corresponding normal cells, e.g., the mRNA or protein is produced at levels at least 25% higher, at least about 50% to about 75% higher, at least about 90% higher, at least about 1.5-fold, at least about 2-fold, or at least about 5-fold that of a corresponding normal cell. In the case of colon cancer it has been observed in about 50% of colon cancer tested express the P-cadherin transcript more than 5-fold that of normal colon cells (based on microarray chip data). The comparison can be made between different tissues or between different cells.

The terms “polynucleotide” and “nucleic acid”, used interchangeably herein, refer to a polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms furhter include, but are not limited to, mRNA or cDNA that comprise intronic sequences (see, e.g., Niwa et al. (1999) Cell 99(7):691-702). The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynuclotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the terms “a gene that is differentially expressed in a cancer cell,” and “a polynucleotide that is differentially expressed in a cancer cell are used interchangeably herein, and generally refer to a polynucleotide that is expressed, at higher levels in cancer cells, e.g., mRNA is found at levels at least about 25%, at least about 50% to about 75%, at least about 90%, at least about 1.5-fold, at least about 2-fold, at least about 5-fold, at least about 10-fold, at least about 95%, or at least about 50-fold or more, different (e.g., higher or lower) in a cancer cell when compared with a cell of the same cell type that is not cancerous. The comparison can be made between two tissues, for example, if one is using in situ hybridization or another assay method that allows some degree of discrimination among cell types in the tissue. The comparison may also be made between cells removed from their tissue source. “P-cadherin” is a gene that is differentially expressed in about 50% colon cancers tested to date.

“Differentially expressed polynucleotide” as used herein refers to a nucleic acid molecule (RNA or DNA) comprising a sequence that represents a differentially expressed gene, e.g., the differentially expressed polynucleotide comprises a sequence (e.g., an open reading frame encoding a gene product; a non-coding sequence) that uniquely identifies a differentially expressed gene so that detection of the differentially expressed polynucleotide in a sample is correlated with the presence of a differentially expressed gene in a sample.

“Differentially expressed polynucleotides” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

As used herein, the term “a polypeptide associated with cancer” refers to a polypeptide encoded by a polynucleotide that is differentially expressed in a cancer cell, e.g., colon cancer.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom, such as colon or another digestive cancer, e.g., stomach or liver, or breast cancer.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and so on.

As used herein the term “isolated” refers to a polynucleotide, a polypeptide, an antibody, or a host cell that is in an environment different from that in which the polynucleotide, the polypeptide, the antibody, or the host cell naturally occurs. A polynucleotide, a polypeptide, an antibody, or a host cell which is isolated is generally substantially purified.

As used herein, the term “substantially purified” refers to a compound (e.g., either a polynucleotide or a polypeptide or an antibody) that is removed from its natural environment and is at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the composition, more preferably at least about 95% or even 99% by weight.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, metastatic, and non-metastatic cells. Detection of cancerous cell is of particular interest.

P-Cadherin Polynucleotides

In one aspect, the present invention relates to the inhibition or detection of a polynucleotide encoding P-cadherin that is differentially expressed in some cancers, particularly some digestive cancers, such as colon cancer. The polynucleotide, as well as polypeptides encoded thereby, find use in a variety of therapeutic and diagnostic methods.

The scope of the invention with respect to polynucleotide compositions useful in the methods described herein includes, but is not necessarily limited to, polynucleotides having a sequence set forth in any one of the polynucleotide sequences provided herein; polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicted.

The invention features P-cadherin polynucleotides that are expressed in human cancer tissues, particularly human colon tissue. Nucleic acid compositions described herein of particular interest comprise a sequence set forth in any one of the polynucleotide sequences provided herein or an identifying sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject nucleic acid compositions include full length cDNAs or mRNAs that encompass an identifying sequence of contiguous nucleotides from any one of the polynucleotide sequences provided herein.

The polynucleotides useful in the methods described herein also include polynucleotides having sequence similarity or sequence identity with native P-cadherin DNA. This includes associated 5′ and 3′ untranslated sequences, promoter and enhancer sequences and sequences in sense or antisense orientation. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

In one embodiment, hybridization is performed using at least 15 contiguous nucleotides (nt) of at least one of the polynucleotide sequences provided herein. That is, when at least 15 contiguous nt of one of the disclosed polynucleotide sequences is used as a probe, the probe will preferentially hybridize with a nucleic acid comprising the complementary sequence, allowing the identification and retrieval of the nucleic acids that uniquely hybridize to the selected probe. Probes from more than one polynucleotide sequences provided herein can hybridize with the same nucleic acid if the cDNA from which they were derived corresponds to one mRNA. Probes of more than 15 nt can be used, e.g., probes of a size within the range of about 18 nt, 25 nt, 50 nt, 75 nt or 100 nt, but in general about 15 nt represents sufficient sequence for unique identification.

Polynucleotides contemplated by the invention also include naturally occurring variants of the nucleotide sequences (e.g., degenerate variants, allelic variants, etc.). Variants of the polynucleotides contemplated by the invention are identified by hybridization of putative variants with nucleotide sequences disclosed herein, preferably by hybridization under stringent conditions. For example, by using appropriate wash conditions, variants of the polynucleotides described herein can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologs corresponding to the P-cadherin polynucleotide sequences provided herein, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity to a P-cadherin gene or portion thereof, preferably the extracellular coding region portion of the gene, e.g., at least 75% sequence identity, usually at least 90%, more usually at least 95%, 96%, 97%, 98% or 99% between nucleotide sequences. Sequence similarity is calculated based on a reference sequence, which may be a subset of a larger sequence, preferably the extracellular coding sequence, e.g. as a conserved motif, part of coding region, flanking region, etc. A reference sequence will usually be at least about 18 contiguous nt long, more usually at least about 30 nt long, and may extend to the complete sequence that is being compared. Algorithms for sequence analysis are known in the art, such as gapped BLAST, described in Altschul, et al. Nucleic Acids Res. (1997) 25:3389-3402.

In general, variants of the P-cadherin polynucleotides described herein have a sequence identity greater than at least about 65%, preferably at least about 75%, more preferably at least about 85%, and can be greater than at least about 90%, 95%, 96%, 98%, 99% or more as determined by the Smith-Waterman homology search algorithm as implemented in MPSRCH program (Oxford Molecular). For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore it should be noted that different species of mRNAs encoded by the same genomic sequence can exist at varying levels in a cell, and detection of these various levels of mRNA species can be indicative of differential expression of the encoded gene product in the cell.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the subject P-cadherin polypeptides or may comprise non-coding sequences, e.g. from the 5′ or 3′ non-coding region of the gene. As noted, these DNAs or RNAs may be in the sense or antisense orientation. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments contemplated by the invention comprise at least about 10, about 15, about 20, about 35, about 50, about 100, about 150 to about 200, about 250 to about 300, or about 350 contiguous nt selected from the polynucleotide provided herein. For the most part, fragments will be of at least 15 nt, usually at least 18 nt or 25 nt, and up to at least about 50 contiguous nt in length or more. In a preferred embodiment, the polynucleotide molecules comprise a contiguous sequence of at least 12 nt selected from any one of the polynucleotide sequences provided herein.

Probes specific to the P-cadherin polynucleotides can be generated using the P-cadherin polynucleotide sequences disclosed herein. The probes are preferably at least about a 12 nt, 15 nt, 16 nt, 18 nt, 20 nt, 22 nt, 24 nt, or 25 nt fragment of a corresponding contiguous sequence any one of the polynucleotide sequences provided herein, and can be less than 2 kb, 1 kb, 0.5 kb, 0.1 kb, or 0.05 kb in length. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein. More preferably, probes are designed based on a contiguous sequence of one of the subject polynucleotides that remain unmasked following application of a masking program for masking low complexity (e.g., XBLAST) to the sequence., i.e., one would select an unmasked region, as indicated by the polynucleotides outside the poly-n stretches of the masked sequence produced by the masking program.

The P-cadherin polynucleotides of the subject invention are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

The P-cadherin polynucleotides described herein can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The nucleic acid compositions described herein can be used to, for example, produce polypeptides, (which may be used to obtain anti-P-cadherin antibodies) as probes for the detection of mRNA in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of any one of the polynucleotide provided herein or variants thereof in a sample. These and other uses are described in more detail infra.

P-Cadherin Polypeptides and Variants Thereof

The polypeptides contemplated by the invention include those encoded by the disclosed P-cadherin polynucleotides, as well as nucleic acids that, by virtue of the degeneracy of the genetic code, are not identical in sequence to the disclosed P-cadherin polynucleotides. Thus, the invention includes within its scope a polypeptide encoded by a polynucleotide having the sequence of any one of the polynucleotide sequences provided herein, or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant P-cadherin polypeptides have a sequence that has at least about 80%, usually at least about 90%, and more usually at least about 95% sequence identity or higher, i.e. 96%, 97%, 98% or 99% sequence identity with a differentially expressed polypeptide described herein, as measured by BLAST 2.0 using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of P-cadherin polypeptides (or fragments thereof) where the homologs are isolated from other species naturally occurring glycosylated P-cadherins include those produced by normal and neoplastic cells, which may exhibit different glycosylated patterns, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 algorithm, with the parameters described supra.

In general, the P-cadherin polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et a/., Protein Eng. (1994) 7:1379), desired metal binding sites (see, e.g., Toma eta/., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants. In particular, fragments will include those that contain the specific domains or epitopes of the P-cadherin protein.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. Preferably, the subject antibodies will comprise at least one human constant domain or a constant domain that exhibits at least about 90-95% sequence identity with a human constant domain, that retains human effector function.

“Antibody fragments” comprise a portion of an intact antibody, preferably comprising the antigen-binding or variable region thereof. Examples of antibody fragments include Fab, Fab′, F(ab′)², and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a P-sheet configuration, connected by three hypervariable regions, which form loops connecting, and in some cases forming part of, the (3 sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′2 fragment that has two antigen-binding sites and is still capable of crosslinking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear at least one free thiol group. F(ab′)Z antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (x) and lambda (k), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The subunit structures and three-dimensional configurations of different classes of immunoglobulins are well known. The present invention embraces the use of antibodies of different isotypes, e.g. IgG1, IgG2, IgG3, IgG4, IgA and IgA2. In some instances, the use of different antibody isotypes may be preferable. For example, if cell depletion is desirable, antibodies of the IgG1 and IgG3 isotype may be preferred. By contrast, if the P-cadherin is significantly expressed by normal cells, it may be preferable to administer antibodies that inhibit cell proliferation but which do not kill cells directly, e.g. of the IgG2 or IgG4 isotype.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore, eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen binding sites. Diabodies are described more fully in, for example, EP 404,097; WO 93/11161; and Hollinger et al., Proc. Nad. Acad. Sci. USA, 90:6444.-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example. The term monoclonal antibody also specifically includes antibodies made by recombinant methods, phage display, single chain antibodies, et al.

The monoclonal antibodies herein specifically include “chimeric” and “humanized” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen binding sequences derived from a non-human primate (e.g. Old World Monkey, such as baboon, rhesus or cynomolgus monkey) and human constant region sequences (US Pat No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Cuff. Op. Struct Biol. 2:593-596 (1992). Another preferred means of making humanized antibodies is disclosed in WO 01/27160 by AME, which application is incorporated by reference in its entirety herein. Preferably, humanized antibodies will comprise a humanized FR that exhibits at least 65% sequence identity with an acceptor (non-human) FR, e.g. murine FR.

The term “hypervariable region” when used herein refers to the amino acid residues of an antibody which are responsible for antigen-binding. The hypervariable region comprises amino acid residues from a “complementarity determining region” or “CDR” (e.g. residues 24-34 (LI), 50-56 (L2) and 89-97 (L3) in the light chain variable domain and 31-35 (HI), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined. An antagonist “which binds” an antigen of interest, e.g. a B cell surface marker, is one capable of binding that antigen with sufficient affinity and/or avidity such that the antagonist is useful as a therapeutic agent for targeting a cell expressing the antigen.

Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcyRII only, whereas monocytes express FcyRI, FcyRII and FcyRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcyRIII and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FCR” are used to describe a receptor that binds to the Fc region of an antibody.

The preferred FcR is a native sequence human FcR. Moreover, a preferred FcR is one which binds an IgG antibody (a gamma receptor) and includes receptors of the FcyRI, FcyRII, and Fcy RIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain. (see Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:33041 (1995). Other FcRs, including those to be identified in the future, are encompassed by the term “FCR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refer to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

An “isolated” antagonist is one which has been identified and separated and/or recovered from a component of its natural environment. Contaminant components of its natural environment are materials which would interfere with diagnostic or therapeutic uses for the antagonist, and may include enzymes, hormones, and other proteinaceous or nonproteinaceous solutes. In preferred embodiments, the antagonist will be purified (1) to greater than 95% by weight of antagonist as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antagonist includes the antagonist in situ within recombinant cells since at least one component of the antagonist's natural environment will not be present. Ordinarily, however, isolated antagonist will be prepared by at least one purification step.

“Mammal” for purposes of treatment refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

The expression “therapeutically effective amount” refers to an amount of the antagonist which is effective for preventing, ameliorating or treating the particular cancer.

The term “cytotoxic agent” as used herein refers to a substance that inhibits or prevents the function of cells and/or causes destruction of cells. The term is intended to include radioactive isotopes (e.g. I¹³¹, Y⁹⁰, Ar²¹¹, P³², Re¹⁸⁸, Re¹⁸⁶, Sm¹⁵³, B²¹² and others), chemotherapeutic agents, and toxins such as small molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembiehin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromoinycins, dactinomycin, daunorubicin, detorubicin, 6-diazo-5-oxo-L-norleucine, doxorubicin, epirubicin, esorubicin, idambicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofrran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and doxetaxel (TAXOTEW, Rh6ne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4 hydroxytamoxifen, trioxifene, keoxifene, onapristone, and toremifene (Fareston); and anti-androgens such as flutamide, nilutamide, bicalutamide, leuprolide, and goserelin; and pharmaceutically acceptable salts, acids or derivatives of any of the above.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons such as interferon-α, -β and -γ; colony stimulating factors (CSFs) such as macrophage-CSF (M-CSF); granulocyte-macrophage-CSF (GM-CSF); and granulocyte-CSF (GCSF); interleukins (ILs) such as IL-1, IL-la, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wihnan, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-contain ing prodrugs, D-amino acid-modified prod rugs, glycosylated prodrugs, (3-lactam-containing prod rugs, option ally substituted phenoxyacetamide-containing prodrugs or optionally substituted phenylacetamide-containing prodrugs, 5 fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “liposome” is a small vesicle composed of various types of lipids, phospholipids and/or surfactant which is useful for delivery of a drug (such as the antagonists disclosed herein and, optionally, a chemotherapeutic agent) to a mammal. The components of the liposome are commonly arranged in a bilayer formation, similar to the lipid arrangement of biological membranes. The term “package insert” is used to refer to instructions customarily included in commercial packages of therapeutic products, that contain information about the indications, usage, dosage, administration, contraindications and/or warnings concerning the use of such therapeutic products.

Production of Antagonists

The methods and articles of manufacture of the present invention use, or incorporate, a P-cadherin antagonist. Accordingly, methods for generating such antagonists will be described here. The P-cadherin to be used for production of, or screening for, antagonist(s) may be, e.g., a soluble form of the antigen or a portion thereof, containing the desired epitope. Alternatively, or additionally, cells expressing P-cadherin on their cell surface can be used to generate, or screen for, antagonist(s).

While the preferred antagonist is an antibody, antagonists other than antibodies are contemplated herein. For example, the antagonist may comprise a small molecule antagonist optionally fused to, or conjugated with, a cytotoxic agent (such as those described herein). Libraries of small molecules may be screened against P-cadherin or P-cadherin expressing cells in order to identify a small molecule which binds to that antigen. The small molecule may further be screened for its antagonistic properties and/or conjugated with a cytotoxic agent.

In particular, the invention contemplates antibodies that bind to specific regions of the P-cadherin protein, which based on the portion of the protein this bind, possess desirable proportions. In this regard, pCDH is a “classical” or Type I cadherin (CDH), a membrane protein requiring calcium for it's adhesive properties. In addition to calcium, cadherins need to form a cis dimer, between molecules on the same cell, prior to forming trans dimers between molecules on adjacent cells.

The structure of a representative Type I cadherin extracellular domain has recently been determined by X-ray crystallography. This new structure, of C-CDH, is noteworthy because it includes the entire, functional extracellular (EC) domain. The extracellular domain is comprised of 5 repeating domains, rigidified by 12 calcium ions bound in the “interdomain” regions. Each domain has a “Greek key” structure, as was seen in previous structures of cadherin EC domain fragments. The whole EC domain is a long, curved or arced structure, with the first EC domain (EC1) distal to the membrane. (See Gumbiner et al., J. Cell Biol., 148:399-403 (2000); Boggon et al., Science 296:1308-1313 (2002); and Chappuis-Flament et al., J Cell Biol, 154:231-243 (2001)) all incorporated by reference in their entirety herein.

The putative trans dimer interface was observed between the Trp2 face of EC1 subunits as the “strand dimer” interface also seen in earlier structures of EC domain fragments. The “strand dimer” is characterized by symmetric binding of Trp2 into a hydrophobic pocket on the opposing molecule.

The putative cis dimer interface was observed between the face of EC1 opposite to Trp2 and the bottom or C-terminal side of EC2. Such interactions can be extended from molecule to molecule, creating an array of parallel EC domains. This is expected to create an avidity effect on the trans dimer binding.

It is anticipated that P-cadherin, as it is in the same family as C-cadherin will possess a similar domain structure. Accordingly, the invention contemplates producing antibodies to P-cadherin that possess at least one of the following binding proportions.

Specifically, antibodies or other antagonists molecules that that disrupt or inhibit PCDH adhesive activity can be accomplished in several ways, including, but not limited to:

1. Producing antibodies that interfere with strand dimer formation. For example, anti-P-cadherin antibodies can be generated which are antibody directed against an epitope including Trp2 or nearby residues in the P-cadherin protein.

2. Also antibodies can be generated that interfere with the cis dimer formation. An example would be antibodies directed against the C-terminal surface of EC2 and the Trp2-distal surface of EC1 in the P-cadherin protein.

3. Also antibodies can be generated that interfere with calcium binding. Antibodies against the interdomain regions potentially will possess this activity.

4. Still further antibodies can be generated that interfere with overall structure such that the relevant domains can't align properly. For this, antibodies potentially can be directed against a variety of regions anywhere in the EC domain.

Antibodies having one or more of the foregoing properties can be identified by screening a population of anti-P-cadherin monoclonal antibodies for those that possess at least one of these proportions.

The antagonist may also be a peptide generated by rational design or by phage display (see, e.g., WO98/35036 published Aug. 13, 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide. Additionally, the antagonist may be an antisense oligonucleotide or ribozyme.

A description follows as to exemplary techniques for the production of the antibody antagonists used in accordance with the present invention.

Polyclonal Antibodies

Polyclonal antibodies are preferably raised in animals by multiple subcutaneous (sc), intraperitoneal (ip) or intramuscular (im) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R1N=C=NR, where R and RI are different alkyl groups. Animals are immunized against the antigen, immunogenic conjugates, or derivatives by combining, e.g., 100 pg or 5 wg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animals, such as a rabbit or hamster, is immunized as herein above described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell [Goding, Monoclonal Antibodies: Principles and Practice, pp.59-103 (Academic Press, 1986)].

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse human heteromyeloma cell lines also have been described for the production of human monoclonal antibodies [Kozbor, J. Immunol., 133:3001 (1984); Brodeur et al., Monoclonal Antibody Production Techniques and Applications, pp. 51-63 (Marcel Dekker, Inc., New York, 1987)].

Culture medium in which hybridoma cells are growing is assayed for the production of monoclonal antibodies having the requisite specificity, e.g. by an in vitro binding assay such as enzyme-linked immunoabsorbent assay (ELISA) or radioimmunoassay (RIA). The location of the cells that express the antibody may be detected by FACS. Thereafter, hybridoma clones may be subcloned by limiting dilution procedures and grown by standard methods (Goding, Monoclonal Antibodies: Principles and Practice, Academic Press (1986) pp. 59-103). Suitable culture media for this purpose include, for example, DMEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Phickthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al, Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen combining site having specificity for a different antigen.

Additionally, recombinant antibodies against P-cadherin can be produced in transgenic animals, e.g., as described in various patents many of which are assigned to Abgenix and Medarex. For example, recombinant antibodies can be expressed in transgenic animals, e.g., rodents as disclosed in any of U.S. Pat. No. 5,877,397, 5,874,299, 5,814,318, 5,789,650, 5,770,429, 5,661,016, 5,633,425, 5,625,126, 5,569,825, 5,545,806, 6,162,963,6,150,584, 6,130,364, 6,114,598, 6,091,001, 5,939,598. Alternatively, recombinant antibodies can be expressed in the milk of transgenic animals as discussed in U.S. Pat. No. 5,849,992 or 5,827,690 which are assigned to Pfarmin, incorporated by reference herein.

Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al, Nature, 332:323-327 (1988); Verhoeyen et al, Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al, J. Immunol, 151:2296 (1993); Chothia et al., J. Mol. Biol, 196:901 (1987)). Another method uses a particular framework region derived from the consensus sequence of all human antibodies of a particular subgroup of light or heavy chains. The same framework may be used for several different humanized antibodies (Carter et al., Proc. Nad. Acad. Sci. USA, 89:4285 (1992); Presta et al., J. Immunol, 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

As an alternative to humanization, human antibodies can be generated. As discussed above, the production of antibodies, particularly human antibodies in transgenic animals is known. For example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Mad. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g. Johnson, Kevin S. and Chiswell, David J., Current Opinion in Structural Biology 3:564-571(1993). Several sources of V-gene segments can be used for phage display. Clackson et al., Nature, 352: 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. Human antibodies may also be generated by in vitro activated B cells (see U.S. Pat. Nos. 5,567,610 and 5,229,275).

Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments [Carter et al., Bio/Technology 10:163-167 (1992)]. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody”, e.g., as described in U.S. Pat. No. 5,641,870 for example. Such linear antibody fragments may be monospecific or bispecific.

Bispecific Antibodies

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CHI) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equivalent amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain.

Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. ImmunoL., 152:5368 (1994). Antibodies with more than two valencies are contemplated. For example, trispecific antibodies can be prepared. Tutt et al. J. Immunol. 147: 60 (1991).

Conjugates and Other Modifications of the Antagonist

The antagonists used in the methods or included in the articles of manufacture herein are optionally conjugated to a cytotoxic or therapeutic agent. Examples include the chemotherapeutic agents described above. Preferable, such chemotherapies will have a established efficacy in treatment of particular cancer.

Conjugates of an antagonist and one or more small molecule toxins, such as a calicheamicin, a maytansine (U.S. Pat. No. 5,208,020), a trichothene, and CC1065 are also contemplated herein. In one embodiment of the invention, the antagonist is conjugated to one or more maytansine molecules (e.g. about 1 to about 10 maytansinemolecules per antagonist molecule). Maytansine may, for example, be converted to May-SS-Me which may be reduced to May-SH3 and reacted with modified antagonist (Chari et al. Cancer Research 52: 127-131 (1992)) to generate a maytansinoid-antagonist conjugate.

Alternatively, the antagonist is conjugated to one or more calicheamicin molecules. The calicheamicin family of antibiotics are capable of producing double-stranded DNA breaks at sub-picomolar concentrations. Structural analogues of calicheamicin are also known. (Hinman et al. Cancer Research 53: 3336-3342 (1993) and Lode et al. Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antagonist conjugated with a compound having nucleolytic activity (e.g. a ribonuclease or a DNA endonuclease such as a deoxyribonuclease; DNase). A variety of radioactive isotopes are available for the production of radioconjugated antagonists. Examples include Y⁹⁰, At²¹¹, Re¹⁸⁶, Re¹⁸⁸, Sm¹⁵³, Bi²¹², P³² and radioactive isotopes of Lu. Conjugates of the antagonist and cytotoxic agent may be made using a variety of bifunctional protein coupling agents such as N-succin imidyl-3-(2-pyridyidith iol) propionate (S PDP), succi n imidyl-4-(N-maleimidomethyl) cyclohexane-l-carboxylate, iminothiolane (IT), bifunctional derivatives of imidoesters (such as dimethyl adipimidate HCL), active esters (such as disuccinimidyl suberate), aidehydes (such as glutareldehyde), bis azido compounds (such as bis (p-azidobenzoyl) hexanediamine), bis-diazonium derivatives (such as bis-(pdiazoniumbenzoyl)-ethylenediamine), diisocyanates (such as tolyene 2,6-diisocyanate), and bis-active fluorine compounds (such as 1,5-difluoro-2, 4-dinitrobenzene). For example, a ricin immunotoxin can be prepared as described in Vitetta et al. Science 238:1098 (1987). Carbon-14-labeled 1-isothiocyanatobenzyl-3-methyidiethylene triaminepentaacetic acid (MX-DTPA) is an exemplary chelating agent for conjugation of radionucleotide to the antagonist. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used. Alternatively, a fusion protein comprising the antagonist and cytotoxic agent may be made, e.g. by recombinant techniques or peptide synthesis.

In yet another embodiment, the antagonist may be conjugated to a “receptor” (such streptavidin) for utilization in tumor pretargeting wherein the antagonist-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). The antagonists of the present invention may also be conjugated with a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate cleaving enzymes such as β-galactosidase and neuraminidase useful for converting glycosylated prodrugs into free drugs; (3-lactamase useful for converting drugs derivatized with (3-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antagonist-abzyme conjugates can be prepared as described herein for delivery of the abzyme to a tumor cell population.

Enzymes can be covalently bound to the P-cadherin antagonist by techniques well known in the art such as the use of the heterobifunctional crosslinking reagents discussed above. Alternatively, fusion proteins comprising at least the antigen binding region of an antagonist of the invention linked to at least a functionally active portion of an enzyme of the invention can be constructed using recombinant DNA techniques well known in the art [see, e.g., Neuberger et al., Nature, 312: 604-608 (1984)].

Other modifications of the antagonist are contemplated herein. For example, the antagonist may be linked to one of a variety of nonproteinaceous polymers, e.g. polyethylene glycol, polypropylene glycol, polyoxyalkylenes, or copolymers of polyethylene glycol and polypropylene glycol. The antagonists disclosed herein may also be formulated as liposomes. Liposomes containing the antagonist are prepared by methods known in the art, such as described in Epstein et al., Proc. Mad. Acad Sci. USA, 82:3688 (1985); Hwang et al., Proc. Natl Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al., J. Biol. Chem. 257: 286-288 (1982) via a disulfide interchange reaction. A chemotherapeutic agent is optionally contained within the liposome. See Gabizon et al. J. National Cancer Inst.81(19)1484 (1989). Amino acid sequence modification(s) of protein or peptide antagonists described herein are contemplated. For example, it may be desirable to improve the binding affinity and/or other biological properties of the antagonist.

Amino acid sequence variants of the antagonist are prepared by introducing appropriate nucleotide changes into the antagonist nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the formal construct, provided that the final construct possesses the desired characteristics. The amino acid changes also may alter post-translational processes of the antagonist, such as changing the number or position of glycosylation sites.

A useful method for the identification of certain residues or regions of the antagonist that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide which increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists include the hypervariable regions, but FR alterations are also contemplated.

Substantial modifications in the biological properties of the antagonist are accomplished by selecting substitutions that differ significantly in their effect on maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchangngg a member of one of these classes for another class. Conservative substitutions involve exchanging of amino acids within the same class.

Any cysteine residue not involved in maintaining the proper conformation of the antagonist also may be substituted, generally with serine, to improve the oxidative stability of the molecule and prevent aberrant cross-linking. Conversely, cysteine bond(s) may be added to the antagonist to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g.6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. By altering is meant deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antagonist is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Nucleic acid molecules encoding amino acid sequence variants of the antagonist are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the antagonist.

It may be desirable to modify the antagonist of the invention with respect to effector function, e.g. so as to enhance antigen-dependent cell-mediated cyotoxicity (ADCC) and/or complement dependent cytotoxicity (CDC) of the antagonist. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunetional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

The present invention also embraces screens for identifying anti-P-cadherin antibodies having desirable therapeutic or diagnostic properties. Particularly, the invention embraces screens that identify antibodies that bind P-cadherin with greater relative affinity than other cadherins, e.g., E-cadherin, C-cadherin, N-cadherin or H-cadherin, e.g., 5/1, 10/1, 15/1, 20/1, 50/1, 100/1 or greater affinity relative to E-cadherin or another cadherin other than P-cadherin. Other screens include assays that identify anti-P-cadherin antibodies which affect proliferation and/or adhesion of tumor cells, ADCC or CDC activity, anti-apoptotic assays, cell cycle checkpoint assays, and in vivo assays in transgenic non-human animals, e.g., mice and other rodents. Also the invention contemplates screens to identify antibodies that bind to desired portions of the protein as described supra, and which possess desired properties, e.g., block calcium binding, block dimer formation, block strand formation and/or interfere with P-cadherin domain alignment. Such antibodies can be identified for populations of antibodies provided against P-cadherin protein or fragments.

Polynucleotide Constructs

Polynucleotide molecules encoding a P-cadherin or fragment or a P-cadherin antagonist such as an antibody can be inserted in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Host Cells

An expression construct encoding P-cadherin or a P-cadherin antagonist can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacteriol 153: 163 (1983); Kurtz et al., Mol. Cell. Biol. 6: 142 (1986); Kunze et al., J Basic Microbiol. 25: 141 (1985); Gleeson et al., J. Gen. Microbiol. 132: 3459 (1986), Roggenkamp et al., Mol. Gen. Genet. 202: 302 (1986)); Das et al., J Bacteriol. 158: 1165 (1984); De Louvencourt et al., J Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5:3376 (1985); U.S. Pat. Nos. 4,837,148; 4,929,555; Beach and Nurse, Nature 300: 706 (1981); Davidow et al., Curr. Genet 10:380 (1985); Gaillardin et al., Curr. Genet. 10:49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen eta/. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761(1985); Gorman et al., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. Nos. 4,767,704; 4,657,866; 4,927,762; 4,560,655; WO 90/103430, WO 87/00195, and U.S. RE No. 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Antisense Oligonucleotides

In certain circumstances, it may be desirable to modulate or decrease the amount of P-cadherin expressed. Thus, in another aspect of the present invention, P-cadherin anti-sense oligonucleotides can be made and a method utilized for diminishing the level of expression of P-cadherin by a cell comprising administering one or more P-cadherin anti-sense oligonucleotides. By P-cadherin anti-sense oligonucleotides reference is made to oligonucleotides that have a nucleotide sequence that interacts through base pairing with a specific complementary nucleic acid sequence involved in the expression of P-cadherin such that the expression of P-cadherin is reduced. Preferably, the specific nucleic acid sequence involved in the expression of P-cadherin is a genomic DNA molecule or mRNA molecule that encodes P-cadherin. This genomic DNA molecule can comprise regulatory regions of the P-cadherin gene, or the coding sequence for mature P-cadherin protein.

The term complementary to a nucleotide sequence in the context of P-cadherin antisense oligonucleotides and methods therefor means sufficiently complementary to such a sequence as to allow hybridization to that sequence in a cell, i.e., under physiological conditions. The P-cadherin antisense oligonucleotides preferably comprise a sequence containing from about 8 to about 100 nucleotides and more preferably the antisense oligonucleotides comprise from about 15 to about 30 nucleotides. The P-cadherin antisense oligonucleotides can also contain a variety of modifications that confer resistance to nucleolytic degradation such as, for example, modified internucleoside linages [Uhlmann and Peyman, Chemical Reviews 90:543-548 (1990); Schneider and Banner, Tetrahedron Lett. 31:335, (1990) which are incorporated by reference], modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

Ribozymes

The invention further embraces the synthesis of ribozymes that inhibit P-cadherin expression. Ribozymes are catalytic RNA molecule with ribonucleic activity that are capable of clearing a single-stranded nucleic acid, such as an mRNA, to which they have a complementary region. Thus, ribozymes (e.g., hammerhead ribozymes) can be used to catalytically clear P-cadherin transcripts to thereby inhibit translation of P-cadherin mRNA. A ribozyme having specificity for P-cadherin can be designed based on the nucleotide sequence of P-cadherin, e.g., the human P-cadherin DNA sequence provided herein. Techniques for synthesizing ribozymes are disclosed in Cech et al., U.S. Pat. No. 4,987,071 and 5,116,742 incorporated by reference. Alternatively, P-cadherin mRNA can be used to select a catalytic RNA having a specific ribonucleic activity from a pool of RNA molecules. (See Bartel and Stostak, J. W., J. Biol. Chem. 1261: 1411-1418(1993)).

Alternatively, P-cadherin expression can be inhibited by targeting nucleotide sequences that are complementary to the regulating region of P-cadherin (promoter, enhancer) to form triple helical structures that prevent transcription in target cells. (See Helene et al., Annal. NY Acad. Sci. 660: 27-36 (1992)).

Pharmaceutical Formulations

Therapeutic formulations of the P-cadherin antagonists in accordance with the present invention are prepared for storage by mixing an antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound. Preferably those with complementary activities that do not adversely affect each other. For example, it may be desirable to further provide a cytotoxic agent, chemotherapeutic agent or cytokine. The effective amount of such other agents depends on the amount of antagonist present in the formulation, the type of cancer treatment, and other factors discussed above. These are generally used in the same dosages and with administration routes as used herein before or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations maybe prepared. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOTTM (injectable microspheres composed of lactic acid glycolic acid copolymer and leuprolide acetate), and poly-D-(−)-3-hydroxybutyric acid.

The formulations to be used for in vivo administration must be sterile. This is readily accomplished by filtration through sterile filtration membranes.

Treatment with the Antagonist

A composition comprising a P-cadherin antagonist, e.g. an antibody, or small molecule will be formulated, dosed, and administered in a fashion consistent with good medical practice. Preferably, the P-cadherin antagonist will be a human, chimeric or humanized anti-P-cadherin antibody scFv, or antibody fragment or an antisense oligonucleotide that inhibits P-cadherin expression. Factors for consideration in this context include the particular cancer being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the antagonist to be administered will be governed by such considerations.

As a general proposition, the therapeutically effective amount of the antagonist administered parenterally per dose will be in the range of about 0.1 to 20 mg/kg of patient body weight per day, with the typical initial range of antagonist used being in the range of about 2 to 10 mg/kg.

As noted above, however, these suggested amounts of antagonist are subject to a great deal of therapeutic discretion. The key factor in selecting an appropriate dose and scheduling is the result obtained, as indicated above.

For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

The antagonist is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration.

In addition, the antagonist may suitably be administered by pulse infusion, e.g., with declining doses of the antagonist. Preferably the dosing is given by injections, most preferably intravenous or subcutaneous injections, depending in part on whether the administration is brief or chronic.

One may administer other compounds, such as cytotoxic agents, chemotherapeutic agents, immunosuppressive agents and/or cytokines with the antagonists herein. The combined administration includes coadministration, using separate formulations or a single pharmaceutical formulation, and consecutive administration in either order, wherein preferably there is a time period while both (or all) active agents simultaneously exert their biological activities.

Aside from administration of protein antagonists to the patient the present application contemplates administration of antagonists by gene therapy. Such administration of nucleic acid encoding the antagonist is encompassed by the expression “administering a therapeutically effective amount of an antagonist”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antagonist is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Nad. Acad. Sci. USA 87:3410-3414 (1990). For review of the currently known gene marking and gene therapy protocols see Anderson et al., Science 256:808-813 (1992). See also WO 93/25673 and the references cited therein.

Diagnosis, Prognosis, Assessment of Therapy (Therametrics), and Management of Cancer

The P-cadherin polynucleotides described herein, as well as their gene products, are of further interest as genetic or biochemical markers (e.g., in blood or tissues) that will detect the earliest changes along the carcinogenesis pathway and/or to monitor the efficacy of various therapies and preventive interventions. For example, the level of expression of P-cadherin can be indicative of a poorer prognosis, and therefore warrant more aggressive chemotherapy or radiotherapy for a patient or vice versa. The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy. Determining expression of P-cadherin and comparing a patients profile with known expression in normal tissue and variants of the disease may allow a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

Measuring P-cadherin expression can be useful to monitor patients having or susceptible to cancer to detect potentially malignant events at a molecular level before they are detectable at a gross morphological level. In addition, P-cadherin polynucleotides, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for metastatic colon cancer can also have clinical implications for stomach cancer or endometrial cancer.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well differentiated or low-grade tumors. The following guidelines are generally used for grading tumors: 1) GX Grade cannot be assessed; 2) G1 Well differentiated; G2 Moderately well differentiated; 3) G3 Poorly differentiated; 4) G4 Undifferentiated. The polynucleotides contemplated by the invention can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Detection of cancer. P-cadherin expression pattern can be used to detect cancer, particularly colon cancer, in a subject. Colorectal cancer is one of the most common neoplasms in humans and perhaps the most frequent form of hereditary neoplasia. Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified, which are summarized as follows: 1) Familial adenomatous polyposis (FAP); 2) Gardner's syndrome; 3) Hereditary nonpolyposis colon cancer (HNPCC); and 4) Familial colorectal cancer in Ashkenazi Jews. The expression of appropriate polynucleotides can be used in the diagnosis, prognosis and management of cancer. Detection of cancer can be determined using expression levels of the P-cadherin sequence alone or in combination with other genes. Determination of the aggressive nature and/or the metastatic potential of a colon cancer can be determined by comparing levels of one or more gene products of the genes corresponding to the polynucleotides described herein, and comparing total levels of another sequence known to vary in cancerous tissue, e.g., expression of p53, DCC, ras, FAP (see, e.g., Fearon ER, et al., Cell (1990) 61(5):759; Hamilton SR et al., Cancer (1993) 72:957; Bodmer W, et al., Nat Genet (1994) 4(3):217; Fearon E R, Ann N Y Acad Sci. (1995) 768:101). For example, development of cancer can be detected by examining the level of expression of P-cadherin corresponding to a polynucleotides described herein to the levels of oncogenes (e.g. ras) or tumor suppressor genes (e.g. FAP or p53). Thus expression of specific marker polynucleotides can be used to discriminate between normal and cancerous colon tissue, to discriminate between cancers with different cells of origin, to discriminate between cancers with different potential metastatic rates, etc. For a review of markers of cancer, see, e.g., Hanahan et al. (2000) Cell 100:57-70.

Treatment of cancer. The invention provides methods for inhibiting growth of cancer cells and/or modulating the adhesion, migration and/or metastasis of cancers characterized by P-cadherin expression. Examples thereof include digestive cancers such as colon cancer, stomach cancer and liver cancer, and other cancers, potentially such as lung cancer and breast cancer. The invention embraces treatment of any cancer where the administration of a P-cadherin antagonist modulates (inhibits) at least one of cancer cell proliferation, cancer cell migration, cancer cell adhesion and metastasis. As shown in the examples, P-cadherin antagonists have been demonstrated to inhibit cancer cell adhesion and to inhibit cancer cell proliferation. Inhibition of adhesion may have a modulatory effect on metastasis by inhibiting the ability of a cancer cell to adhere to and develop a tumor at a site different from the original tumor. In general, the methods comprise contacting a cancer cell with a substance that modulates (1) expression of a polynucleotide corresponding to P-cadherin; or (2) a level of and/or an activity of a P-cadherin polypeptide. The methods provide for decreasing the expression of P-cadherin in a cancer cell or decreasing the level of and/or decreasing an activity of a P-cadherin. This inhibition will result in decreased cancer cell proliferation, migration and/or adhesion.

“Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with colon cancer (e.g., CEA, CA19-9, and LASA).

The present invention in particular provides methods for treating P-cadherin associated cancer, preferably colon cancer, comprising administering to an individual in need thereof a substance that reduces cancer cell growth, in an amount sufficient to reduce cancer cell growth and treat the cancer. Whether a substance, or a specific amount of the substance, is effective in treating cancer in patients can be assessed using any of a variety of known diagnostic assays for cancer, including, but not limited to, sigmoidoscopy, proctoscopy, rectal examination, colonoscopy with biopsy, contrast radiographic studies, CAT scans, angiography, and detection of a tumor marker associated with colon cancer in the blood of the individual. The substance can be administered systemically or locally. Thus, in some embodiments, the substance is administered locally, and colon cancer growth is decreased at the site of administration. Local administration may be useful in treating, e.g., a solid tumor.

Diagnostic and Other Methods Involving Detection of P-Cadherin

The present invention provides methods of using the polynucleotides described herein. In specific non-limiting embodiments, the methods are useful for detecting P-cadherin associated cancer cells, especially colon cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of levels of P-cadherin in a cell, e.g., colon cancer cell and/or detection of a P-cadherin polypeptide in a cancer cell. The detection methods of the invention can be conducted in vitro or in vivo, on isolated cells, or in whole tissues or a bodily fluid, e.g., blood, plasma, serum, urine, and the like).

The detection methods can be provided as part of a kit. Thus, the invention further provides kits for detecting the presence and/or a level of a P-cadherin expressed in a cancer cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a colon cancer cell comprise a moiety that specifically binds the polypeptide, which may be a specific antibody. The kits of the invention for detecting a polynucleotide that is differentially expressed in a colon cancer cell comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information. Detecting a P-cadherin polypeptide in a colon cancer cell

In some embodiments, methods are provided for detecting P-cadherin associated cancer by detecting an overexpressing P-cadherin cell. Any of a variety of known methods can be used for detection, including, but not limited to, immunoassay, using antibody specific for the encoded polypeptide, e.g., by enzyme-linked immunosorbent assay (ELISA), radioimmunoassay (RIA), and the like; and functional assays for the encoded polypeptide, e.g., binding activity or enzymatic activity.

For example, an immunofluorescence assay can be easily performed on cells without first isolating the encoded polypeptide. The cells are first fixed onto a solid support, such as a microscope slide or microtiter well. This fixing step can permeabilize the cell membrane. The permeablization of the cell membrane permits the polypeptide-specific antibody to bind. Next, the fixed cells are exposed to an antibody specific for the encoded polypeptide. To increase the sensitivity of the assay, the fixed cells may be further exposed to a second antibody, which is labeled and binds to the first antibody, which is specific for the encoded polypeptide. Typically, the secondary antibody is detectably labeled, e.g., with a fluorescent marker. The cells which express the encoded polypeptide will be fluorescently labeled and easily visualized under the microscope. See, for example, Hashido et al. (1992) Biochem. Biophys. Res. Comm. 187:1241-1248.

As will be readily apparent to the ordinarily skilled artisan upon reading the present specification, the detection methods and other methods described herein can be readily varied. Such variations are within the intended scope of the invention. For example, in the above detection scheme, the probe for use in detection can be immobilized on a solid support, and the test sample contacted with the immobilized probe. Binding of the test sample to the probe can then be detected in a variety of ways, e.g., by detecting a detectable label bound to the test sample to facilitate detected of test sample-immobilized probe complexes.

The present invention further provides methods for detecting the presence of and/or measuring a level of P-cadherin polypeptide in a biological sample, using an antibody specific for P-cadherin. The methods generally comprise: a) contacting the sample with an antibody specific for a P-cadherin; and b) detecting binding between the antibody and molecules of the sample.

Detection of specific binding of the antibody specific for P-cadherin, when compared to a suitable control, is an indication that P-cadherin is present in the sample. Suitable controls include a sample known not to contain P-cadherin; and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, ∃-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where P-cadherin associated cancer cells are present. In these embodiments, a detectably-labeled moiety, e.g., an antibody, which is specific for P-cadherin administered to an individual (e.g., by injection), and labeled cells are located using standard imaging techniques, including, but not limited to, magnetic resonance imaging, computed tomography scanning, and the like. In this manner, P-cadherin expressing cells are differentially labeled.

Detecting a P-Cadherin Polynucleotide in a Cancer Cell

Methods are provided for detecting a P-cadherin cancer cell by detecting expression in the cell of a P-cadherin transcript in a cancer cell. Any of a variety of known methods can be used for detection, including, but not limited to, detection of a transcript by hybridization with a polynucleotide that hybridizes to a P-cadherin polynucleotide; detection of a transcript by a polymerase chain reaction using specific oligonucleotide primers; in situ hybridization of a cell using as a probe a polynucleotide that hybridizes to a gene that is differentially expressed in a colon cancer cell. The methods can be used to detect and/or measure mRNA levels P-cadherin gene expressed in a cancer cell. In some embodiments, the methods comprise: a) contacting a sample with a P-cadherin polynucleotide under conditions that allow hybridization; and b) detecting hybridization, if any.

Detection of differential hybridization, when compared to a suitable control, is an indication of the presence in the sample of a polynucleotide that is differentially expressed in a cancer cell. Appropriate controls include, for example, a sample which is known not to contain a P-cadherin polynucleotide. Conditions that allow hybridization are known in the art. Detection can also be accomplished by any known method, including, but not limited to, in situ hybridization, PCR (polymerase chain reaction), RT-PCR (reverse transcription-PCR), and “Northern” or RNA blotting, or combinations of such techniques, using a suitably labeled polynucleotide. A variety of labels and labeling methods for polynucleotides are known in the art and can be used in the assay methods of the invention. Specific hybridization can be determined by comparison to appropriate controls.

Polynucleotide generally comprising at least 12 contiguous nt of the P-cadherin polynucleotide provided herein, as shown in the Sequence Listing, are used for a variety of purposes, such as probes for detection of and/or measurement of, transcription levels of a polynucleotide that is differentially expressed in a colon cancer cell. A probe that hybridizes specifically to a polynucleotide disclosed herein should provide a detection signal at least 5-, 10-, or 20-fold higher than the background hybridization provided with other unrelated sequences. It should be noted that “probe” as used herein is meant to refer to a polynucleotide sequence used to detect a P-cadherin gene product in a test sample. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophoress, and enzymes. Other examples of nucleotide hybridization assays are described in WO92/02526 and U.S. Pat. No. 5,124,246.

PCR is another means for detecting small amounts of target nucleic acids (see, e.g., Mullis et al., Meth. Enzymol. (1987) 155:335; U.S. Pat. Nos. 4,683,195; and 4,683,202). Two primer polynucleotides nucleotides that hybridize with the target nucleic acids are used to prime the reaction. The primers can be composed of sequence within or 3′ and 5′ to the polynucleotides of the Sequence Listing. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp.14.2-14.33. A detectable label may be included in the amplification reaction. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The label may be a two stage system, where the polynucleotides is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Arrays

Polynucleotide arrays provide a high throughput technique that can assay a large number of polynucleotides or polypeptides in a sample. This technology can be used as a tool to test for differential expression. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci U S A. 93(20):10614-9; Schena et al. (1995) Science 270(5235):467-70; Shalon et al. (1996) Genome Res. 6(7):639A5, U.S. Pat. No. 5,807,522, EP 799 897; WO 97/29212; WO 97/27317; EP 785 280; WO 97/02357; U.S. Pat. Nos. 5,593,839; 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of a P-cadherin gene, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

A preferred nucleotide for use in selecting P-cadherin arrays has the sequence below, which was obtained from Incyte (SEQ ID NO:3):

| | |
|---|---|
| gcggtgacga cggggaccat tttaccatca ccacccaccc tgagagcaac cagggcatcc | 60 |
| tgacaaccag gaagggtttg gattttgagg ccaaaaacca gcacaccctg tacgttgaag | 120 |
| tgaccaacga ggcccctttt gtgctgaagc tcccaacctc | 160 |

Articles of Manufacture

In another embodiment of the invention, an article of manufacture containing P-cadherin useful for the treatment of the diseases or disorders described above is provided. The article of manufacture comprises a container and a label or package insert on or associated with the container. Suitable containers include, for example, bottles, vials, syringes, etc. The containers maybe formed from a variety of materials such as glass or plastic. The container holds or contains a composition which is effective for treating the disease or disorder of choice and may have a sterile access port (for example the container may be an intravenous solution bag or a vial having a stopper pierceable by a hypodermic injection needle). At least one active agent in the composition a P-cadherin antagonist, preferably an antibody. The label or package insert indicates that the composition is used for treating a patient having or predisposed to cancer, e.g., colon cancer. The article of manufacture may further comprise a second container comprising a pharmaceutically-acceptable diluent buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Further details of the invention are illustrated by the following non-limiting Examples. The disclosure of all citations in the specification are expressly incorporated by reference.

### EXAMPLES

### Example 1

### Source of Biological Materials

The biological materials used in the experiments that led to the present invention are described below.

Source of Patient Tissue Samples

Normal and cancerous tissues were collected from patients using laser capture microdissection (LCM) techniques, which are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001). Table 1 (supra) provides information about each patient from which the samples were isolated, including: the Patient Id. and Path Report ID, numbers assigned to the patient and the pathology reports for identification purposes; the anatomical location of the tumor (AnatomicalLoc); The Primary Tumor Size; the Primary Tumor Grade; the Histopathologic Grade; a description of local sites to which the tumor had invaded (Local Invasion); the presence of lymph node metastases (Lymph Node Metastasis); incidence of lymph node metastases (provided as number of lymph nodes positive for metastasis over the number of lymph nodes examined) (Incidence Lymphnode Metastasis); the Regional Lymphnode Grade; the identification or detection of metastases to sites distant to the tumor and their location (Distant Met & Loc); a description of the distant metastases (Description Distant Met); the grade of distant metastasis (Distant Met Grade); and general comments about the patient or the tumor (Comments). Adenoma was not described in any of the patients. Adenoma dysplasia (described as hyperplasia by the pathologist) was described in Patient ID No. 695. Extranodal extensions were described in two patients, Patient ID Nos. 784 and 791. Lymphovascular invasion was described in seven patients, Patient ID Nos. 128, 278, 517, 534, 784, 786, and 791. Crohn's-like infiltrates were described in seven patients, Patient ID Nos. 52, 264, 268, 392, 393, 784, and 791.

TABLE 1

| T/N | | | | | | | | Inci- | | | | | | | | Lymph- | | | |
|---|---|---|---|---|---|---|---|---|---|---|---|---|---|---|---|---|---|---|---|
| micro- | | | | | | Dis- | Descrip- | dence | | | Re- | | | | | node | | | |
| array | | | | | Dis- | tant | tion | Lymph- | | Pri- | gion- | Dis- | | | | vas- | Extra- | | |
| ratio | Pa- | Path | | | tant | Met | Dis- | node | Anato- | mary | al | tant | | Histo- | | cular | nodal | Crohn's | |
| P- | tient | Rep | Local | Ln | Meta- | Loca- | tant | Meta- | mical | Tumor | LN | Met | | path | Tumor | Inva- | Exten- | like | |
| cadh | ID | ID | Invasion | Met | siasis | tion | Met | stasis | Loc | Grade | Grade | Grade | Group | Grade | Size | sion | sion | Int. | Comment |
| 2.39 | 15 | 21 | Extending into | pos | negative | | | 3/8 | Ascend- | T3 | N1 | MX | III | G2 | 4.0 | Not | negative | neg | invasive |
| | | | subserosal | | | | | | ing colon | | | | | | | identified | | | adenocarcinoma, |
| | | | adipose tissue | | | | | | | | | | | | | | | | moderately |
| | | | | | | | | | | | | | | | | | | | differentiated; focal |
| | | | | | | | | | | | | | | | | | | | perineural invasion |
| | | | | | | | | | | | | | | | | | | | is seen |
| 3.01 | 52 | 71 | Invasion | neg | negative | | | 0/12 | Ascend- | T3 | N0 | M0 | II | G3 | 9.0 | Not | negative | pos | Hyperplastic polyp |
| | | | through | | | | | | ing colon | | | | | | | identified | | | in appendix. |
| | | | muscularis | | | | | | | | | | | | | | | | |
| | | | propria, | | | | | | | | | | | | | | | | |
| | | | subserosal | | | | | | | | | | | | | | | | |
| | | | involvement; | | | | | | | | | | | | | | | | |
| | | | ileocec. valve | | | | | | | | | | | | | | | | |
| | | | involvement | | | | | | | | | | | | | | | | |
| 6.82 | 121 | 140 | Invasion of | neg | negative | | | 0/34 | Sigmoid | T4 | N0 | M0 | II | G2 | 6 | Not | negative | neg | Perineural invasion; |
| | | | muscularis | | | | | | | | | | | | | identified | | | donut anastomosis |
| | | | propria into | | | | | | | | | | | | | | | | negative. One |
| | | | serosa, | | | | | | | | | | | | | | | | tubulovillous and |
| | | | involving | | | | | | | | | | | | | | | | one tubular |
| | | | submucosa of | | | | | | | | | | | | | | | | adenoma with no |
| | | | urinary bladder | | | | | | | | | | | | | | | | high grade |
| | | | | | | | | | | | | | | | | | | | dysplasia. |
| 6.56 | 125 | 144 | Invasion | neg | negative | | | 0/19 | Cecum | T3 | N0 | M0 | II | G2 | 6 | Not | negative | neg | patient history of |
| | | | through the | | | | | | | | | | | | | identified | | | metastatic |
| | | | muscularis | | | | | | | | | | | | | | | | melanoma |
| | | | propria into | | | | | | | | | | | | | | | | |
| | | | suserosal | | | | | | | | | | | | | | | | |
| | | | adipose tissue. | | | | | | | | | | | | | | | | |
| | | | lleocecal | | | | | | | | | | | | | | | | |
| | | | junction. | | | | | | | | | | | | | | | | |
| 2.29 | 128 | 147 | Invasion of | pos | Nega- | | | 1/5 | Transverse | T3 | N1 | M0 | III | G2 | 5.0 | Identified | negative | neg | |
| | | | muscularis | | tive | | | | colon | | | | | | | | | | |
| | | | propria into | | | | | | | | | | | | | | | | |
| | | | percolonic fat | | | | | | | | | | | | | | | | |
| 2.15 | 130 | 149 | Through wall | pos | negative | | | 10/24 | Splenic | T3 | N2 | M1 | | | 5.5 | Not | negative | neg | |
| | | | and into | | | | | | flexure | | | | | | | identified | | | |
| | | | surrounding | | | | | | | | | | | | | | | | |
| | | | adipose tissue | | | | | | | | | | | | | | | | |
| 4.51 | 133 | 152 | Invasion | neg | negative | | | 0/9 | Rectum | T3 | N0 | M0 | II | G2 | 50 | Not | negative | neg | Small separate |
| | | | through | | | | | | | | | | | | | identified | | | tubular adenoma |
| | | | muscularis | | | | | | | | | | | | | | | | (0.4 cm) |
| | | | propria into | | | | | | | | | | | | | | | | |
| | | | non- | | | | | | | | | | | | | | | | |
| | | | peritonealized | | | | | | | | | | | | | | | | |
| | | | pericolic tissue; | | | | | | | | | | | | | | | | |
| | | | gross | | | | | | | | | | | | | | | | |
| | | | configuration is | | | | | | | | | | | | | | | | |
| | | | annular. | | | | | | | | | | | | | | | | |
| 2.50 | 141 | 160 | Invasion of | pos | positive | Liver | adeno- | 7/21 | Cecum | T3 | N2 | M1 | IV | G2 | 5.5 | Not | negative | neg | Perineural invasion |
| | | | muscularis | | | | carcinoma | | | | | | | | | identified | | | identified adjacent |
| | | | propria into | | | | consis- | | | | | | | | | | | | to metastatic |
| | | | pericolonic | | | | tant with | | | | | | | | | | | | adenocarcinoma. |
| | | | adipose tissue, | | | | primary | | | | | | | | | | | | |
| | | | but not through | | | | | | | | | | | | | | | | |
| | | | serosa. Arising | | | | | | | | | | | | | | | | |
| | | | from tubular | | | | | | | | | | | | | | | | |
| | | | adenoma. | | | | | | | | | | | | | | | | |
| 11.03 | 156 | 175 | Invasion | pos | negative | | | 2/13 | Hepatic | T3 | N1 | M0 | III | G2 | 3.8 | Not | negative | neg | Separate |
| | | | through | | | | | | flexure | | | | | | | identified | | | tubolovillous and |
| | | | mucsularis | | | | | | | | | | | | | | | | tubular adenomas |
| | | | propria into | | | | | | | | | | | | | | | | |
| | | | subserosa/peric | | | | | | | | | | | | | | | | |
| | | | olic adipose, no | | | | | | | | | | | | | | | | |
| | | | serosal | | | | | | | | | | | | | | | | |
| | | | involvement. | | | | | | | | | | | | | | | | |
| | | | Gross | | | | | | | | | | | | | | | | |
| | | | configuration | | | | | | | | | | | | | | | | |
| | | | annular. | | | | | | | | | | | | | | | | |
| 4.69 | 228 | 247 | Invasion | pos | negative | | | 1/8 | Rectum | T3 | N1 | MX | III | G2 to | 5.8 | Identified | negative | neg | Hyperplastic polyps |
| | | | through | | | | | | | | | | | G3 | | | | | |
| | | | muscularis | | | | | | | | | | | | | | | | |
| | | | propria to | | | | | | | | | | | | | | | | |
| | | | involve | | | | | | | | | | | | | | | | |
| | | | subserosal, | | | | | | | | | | | | | | | | |
| | | | perirectoal | | | | | | | | | | | | | | | | |
| | | | adipose, and | | | | | | | | | | | | | | | | |
| | | | serosa | | | | | | | | | | | | | | | | |
| 17.06 | 264 | 283 | Invasion | neg | negative | | | 0/10 | Ascending | T3 | N0 | M0 | II | G2 | 5.5 | Not | negative | pos | Tubulovillous |
| | | | through | | | | | | colon | | | | | | | identified | | | adenoma with high |
| | | | muscularis | | | | | | | | | | | | | | | | grade dysplasia |
| | | | propria into | | | | | | | | | | | | | | | | |
| | | | subserosal | | | | | | | | | | | | | | | | |
| | | | adipose tissue. | | | | | | | | | | | | | | | | |
| 14.57 | 266 | 285 | Invades | neg | positive | Mesen- | 0.4 cm, | 0/15 | Transverse | T3 | N1 | MX | III | G2 | 9 | Not | negative | neg | |
| | | | through | | | teric | may | | colon | | | | | | | identified | | | |
| | | | muscularis | | | deposit | represent | | | | | | | | | | | | |
| | | | propria to | | | | lymph | | | | | | | | | | | | |
| | | | involve | | | | node | | | | | | | | | | | | |
| | | | pericolonic | | | | com- | | | | | | | | | | | | |
| | | | adipose, | | | | pletely | | | | | | | | | | | | |
| | | | extends to | | | | replaced | | | | | | | | | | | | |
| | | | serosa. | | | | by tumor | | | | | | | | | | | | |
| 4.28 | 268 | 287 | Invades full | neg | negative | | | 0/12 | Cecum | T2 | N0 | M0 | I | G2 | 6.5 | Not | negative | pos | |
| | | | thickness of | | | | | | | | | | | | | identified | | | |
| | | | muscularis | | | | | | | | | | | | | | | | |
| | | | propria, but | | | | | | | | | | | | | | | | |
| | | | mesenteric | | | | | | | | | | | | | | | | |
| | | | adipose free of | | | | | | | | | | | | | | | | |
| | | | malignancy | | | | | | | | | | | | | | | | |
| 3.67 | 278 | 297 | Invasion into | pos | negative | | | 7/10 | Rectum | T3 | N2 | M0 | III | G2 | 4 | Identified | negative | neg | Descending colon |
| | | | perirectal | | | | | | | | | | | | | | | | polyps, no HGD or |
| | | | adipose tissue. | | | | | | | | | | | | | | | | carcinoma |
| | | | | | | | | | | | | | | | | | | | identified. |
| 9.22 | 295 | 314 | Invasion | neg | negative | | | 0/12 | Ascending | T3 | N0 | M0 | II | G2 | 5.0 | Not | negative | neg | Melanosis coli and |
| | | | through | | | | | | colon | | | | | | | identified | | | diverticular disease. |
| | | | muscularis | | | | | | | | | | | | | | | | |
| | | | propria into | | | | | | | | | | | | | | | | |
| | | | percolic | | | | | | | | | | | | | | | | |
| | | | adipose tissue. | | | | | | | | | | | | | | | | |
| 3.29 | 339 | 358 | Extends into | neg | negative | | | 0/6 | Rectosig- | T3 | N0 | M0 | II | G2 | 6 | Not | negative | neg | 1 hyperplastic polyp |
| | | | perirectal fat | | | | | | moid | | | | | | | identified | | | identified |
| | | | but does not | | | | | | | | | | | | | | | | |
| | | | reach serosa | | | | | | | | | | | | | | | | |
| 3.77 | 341 | 360 | Invasion | neg | negative | | | 0/4 | Ascending | T3 | N0 | MX | II | G2 | 2 cm | Not | negative | neg | |
| | | | through | | | | | | colon | | | | | | in- | identified | | | |
| | | | muscularis | | | | | | | | | | | | va- | | | | |
| | | | propria to | | | | | | | | | | | | sive | | | | |
| | | | involve | | | | | | | | | | | | | | | | |
| | | | pericolonic fat. | | | | | | | | | | | | | | | | |
| | | | Arising from | | | | | | | | | | | | | | | | |
| | | | villous | | | | | | | | | | | | | | | | |
| | | | adenoma. | | | | | | | | | | | | | | | | |
| 2.75 | 356 | 375 | Through colon | neg | negative | | | 0/4 | Sigmoid | T3 | N0 | M0 | II | G2 | 6.5 | Not | negative | neg | |
| | | | wall into | | | | | | | | | | | | | identified | | | |
| | | | subserosal | | | | | | | | | | | | | | | | |
| | | | adipose tissue. | | | | | | | | | | | | | | | | |
| | | | No serosal | | | | | | | | | | | | | | | | |
| | | | spread seen. | | | | | | | | | | | | | | | | |
| 1.69 | 360 | 412 | Invasion thru | pos | negative | | | 1/5 | Ascending | T3 | N1 | M0 | III | G2 | 4.3 | Not | negative | neg | Two mucosal |
| | | | muscularis | | | | | | colon | | | | | | | identified | | | polyps |
| | | | propria to | | | | | | | | | | | | | | | | |
| | | | pericolonic fat | | | | | | | | | | | | | | | | |
| 2.16 | 392 | 444 | Invasion | pos | positive | Liver | Macro- | 1/6 | Ascending | T3 | N1 | M1 | IV | G2 | 2 | Not | negative | pos | Tumor arising at |
| | | | through | | | | vesicular | | colon | | | | | | | identified | | | prior ileocolic |
| | | | muscularis | | | | and | | | | | | | | | | | surgical | |
| | | | propria into | | | | micro- | | | | | | | | | | | | anastomosis. |
| | | | subserosal | | | | vesicular | | | | | | | | | | | | |
| | | | adipose tissue, | | | | steatosis | | | | | | | | | | | | |
| | | | not serosa. | | | | | | | | | | | | | | | | |
| 3.88 | 393 | 445 | Cecum, | neg | negative | | | 0/21 | Cecum | T3 | N0 | M0 | II | G2 | 6.0 | Not | negative | pos | |
| | | | invades | | | | | | | | | | | | | identified | | | |
| | | | through | | | | | | | | | | | | | | | | |
| | | | muscularis | | | | | | | | | | | | | | | | |
| | | | propria to | | | | | | | | | | | | | | | | |
| | | | involve | | | | | | | | | | | | | | | | |
| | | | subserosal | | | | | | | | | | | | | | | | |
| | | | adipose tissue | | | | | | | | | | | | | | | | |
| | | | but not serosa. | | | | | | | | | | | | | | | | |
| 2.88 | 413 | 465 | Invasive | neg | positive | Liver | adeno- | 0/7 | Ascending | T3 | N0 | M1 | IV | G2 | 4.8 | Not | negative | neg | rediagnosis of |
| | | | through | | | | carcinoma | | colon | | | | | | | identified | | | oophorectomy path |
| | | | muscularis to | | | | in | | | | | | | | | | | | to metastatic colon |
| | | | involve | | | | multiple | | | | | | | | | | | | cancer. |
| | | | periserosal fat; | | | | slides | | | | | | | | | | | | |
| | | | abutting | | | | | | | | | | | | | | | | |
| | | | ileocecal | | | | | | | | | | | | | | | | |
| | | | junction. | | | | | | | | | | | | | | | | |
| 6.55 | 505 | 383 | Invasion | pos | positive | Liver | moderate- | 2/17 | | T3 | N1 | M1 | IV | G2 | 7.5 | Not | negative | neg | Anatomical location |
| | | | through | | | | ly | | | | | | | | cm | identified | | | of primary not |
| | | | muscularis | | | | differen- | | | | | | | | max | | | | notated in report. |
| | | | propria | | | | tiated | | | | | | | | dim | | | | Evidence of chronic |
| | | | involving | | | | adeno- | | | | | | | | | | | | colitis. |
| | | | pericolic | | | | carcinoma | | | | | | | | | | | | |
| | | | adipose, | | | | consistent | | | | | | | | | | | | |
| | | | serosal surface | | | | primary | | | with | | | | | | | | | |
| | | | uninvolved | | | | | | | | | | | | | | | | |
| 6.52 | 517 | 395 | Penetrates | pos | negative | | | 6/6 | Sigmoid | T3 | N2 | M0 | IV | G2 | 3 | Identified | negative | neg | No mention of |
| | | | muscularis | | | | | | | | | | | | | | | | distant met in report |
| | | | propria, | | | | | | | | | | | | | | | | |
| | | | involves | | | | | | | | | | | | | | | | |
| | | | pericolonic fat. | | | | | | | | | | | | | | | | |
| 2.40 | 534 | 553 | Invasion | neg | negative | | | 0/8 | Ascend- | T3 | N0 | M0 | II | G3 | 12 | Identified | negative | neg | Omentum with |
| | | | through the | | | | | | ing | | | | | | | | | | fibrosis and fat |
| | | | muscularis | | | | | | colon | | | | | | | | | | necrosis. Small |
| | | | propria | | | | | | | | | | | | | | | | bowel with acute |
| | | | involving | | | | | | | | | | | | | | | | and chronic |
| | | | pericolic fat. | | | | | | | | | | | | | | | | serositis, focal |
| | | | Serosa free of | | | | | | | | | | | | | | | | abscess and |
| | | | tumor. | | | | | | | | | | | | | | | | adhesions. |
| 2.01 | 546 | 565 | Invasion | pos | positive | Liver | metastatic | 6/12 | Ascending | T3 | N2 | M1 | IV | G2 | 5.5 | Not | negative | neg | |
| | | | through | | | | adeno- | | colon | | | | | | | identified | | | |
| | | | muscularis | | | | carcinoma | | | | | | | | | | | | |
| | | | propria | | | | | | | | | | | | | | | | |
| | | | extensively | | | | | | | | | | | | | | | | |
| | | | through | | | | | | | | | | | | | | | | |
| | | | submucosal | | | | | | | | | | | | | | | | |
| | | | and extending | | | | | | | | | | | | | | | | |
| | | | to serosa. | | | | | | | | | | | | | | | | |
| 4.04 | 577 | 596 | Invasion | neg | negative | | | 0/58 | Cecum | T3 | N0 | M0 | II | G2 | 11.5 | Not | negative | neg | Appendix dilated |
| | | | through the | | | | | | | | | | | | | identified | | | and fibrotic, but not |
| | | | bowel wall, into | | | | | | | | | | | | | | | | involved by tumor |
| | | | suberosal | | | | | | | | | | | | | | | | |
| | | | adipose. | | | | | | | | | | | | | | | | |
| | | | Serosal surface | | | | | | | | | | | | | | | | |
| | | | free of tumor. | | | | | | | | | | | | | | | | |
| 0.00 | 695 | 714 | Extending | neg | negative | | | 0/22 | Cecum | T3 | N0 | MX | II | G2 | 14 | Not | negative | neg | tubular adenoma |
| | | | through bowel | | | | | | | | | | | | | identified | | | and hyperplstic |
| | | | wall into | | | | | | | | | | | | | | | | polyps present, |
| | | | serosal fat | | | | | | | | | | | | | | | | moderately |
| | | | | | | | | | | | | | | | | | | | differentiated |
| | | | | | | | | | | | | | | | | | | | adenoma with |
| | | | | | | | | | | | | | | | | | | | mucinous |
| | | | | | | | | | | | | | | | | | | | diferentiation (% not |
| | | | | | | | | | | | | | | | | | | | stated) |
| 2.33 | 784 | 803 | Through | pos | positive | Liver | | 5/17 | Ascending | T3 | N2 | M1 | IV | G3 | 3.5 | Identified | positive | pos | invasive poorly |
| | | | muscularis | | | | | | colon | | | | | | | | | | differentiated |
| | | | propria into | | | | | | | | | | | | | | | | adenosquamous |
| | | | pericolic soft | | | | | | | | | | | | | | | | carcinoma |
| | | | tissues | | | | | | | | | | | | | | | | |
| 2.62 | 786 | 805 | Through | neg | positive | Liver | | 0/12 | Des- | T3 | N0 | M1 | IV | G2 | 9.5 | Identified | negative | neg | moderately |
| | | | muscularis | | | | | | cending | | | | | | | | | | differentiated |
| | | | propria into | | | | | | colon | | | | | | | | | | invasive |
| | | | pericolic fat, but | | | | | | | | | | | | | | | | |
| | | | not at serosal | | | | | | | | | | | | | | | | |
| | | | surface | | | | | | | | | | | | | | | | |
| 4.30 | 791 | 810 | Through the | pos | positive | Liver | | 13/25 | Ascending | T3 | N2 | M1 | IV | G3 | 5.8 | Identified | positive | pos | poorly differentiated |
| | | | muscularis | | | | | | colon | | | | | | | | | | invasive colonic |
| | | | propria into | | | | | | | | | | | | | | | | adenocarcinoma |
| | | | pericolic fat | | | | | | | | | | | | | | | | |
| 3.64 | 888 | 908 | Into muscularis | pos | positive | Liver | | 3/21 | Ascending | T2 | N0 | M1 | IV | G1 | 2.0 | Not | negative | neg | well-to moderately- |
| | | | propria | | | | | | colon | | | | | | | identified | | | differentiated |
| | | | | | | | | | | | | | | | | | | | adenocarcinoma; |
| | | | | | | | | | | | | | | | | | | | this patient has |
| | | | | | | | | | | | | | | | | | | | tumors of the |
| | | | | | | | | | | | | | | | | | | | ascending colon and |
| | | | | | | | | | | | | | | | | | | | the sigmoid colon |
| 2.30 | 889 | 909 | Through | pos | positive | Liver | | 1/4 | Cecum | T3 | N1 | M1 | IV | G2 | 4.8 | Not | negative | neg | moderately |
| | | | muscularis | | | | | | | | | | | | | identified | | | differentiated |
| | | | propria int | | | | | | | | | | | | | | | | adenocarcinoma |
| | | | subserosal | | | | | | | | | | | | | | | | |
| | | | tissue | | | | | | | | | | | | | | | | |

### Example 2

### P-Cadherin Microarray Design

P-Cadherin microarrays were constructed using the oligonucleotide (SEQ ID NO:3) previously identified as a probe having identical spatial layout and control-spot set with each array divided into two areas, each area having twelve groupings of 32×12 spots, for a total of about 9,216 spots per array. The two areas are spotted identically, providing for at least two duplicates of each clone per array. Spotting was accomplished using PCR amplified products from 0.5 kb to 2.0 kb and spotted using a Molecular Dynamics Gen III spotter according to the manufacturer's recommendations. The first row of each of the 24 groupings on the array had about 32 control spots, including 4 negative control spots and 8 test polynucleotides.

The test polynucleotides were spiked into each sample before the labeling reaction, with a range of concentrations from 2-600 pg/slide and ratios of 1:1. For each array design, two slides were hybridized with the test samples reverse-labeled in the labeling reaction. This provided for about 4 duplicate measurements for each clone, two of one color and two of the other, for each sample.

### Example 3

### Identification of Differentially Expressed Genes

cDNA probes were prepared from total RNA isolated from the patient cells described in Example 1. Since LCM provides for the isolation of specific cell types to provide a substantially homogeneous cell sample, this provided for a similarly pure RNA sample.

Total RNA was first reverse transcribed into cDNA using an oligodT primer containing the T7 RNA polymerase promoter, followed by second strand DNA synthesis. cDNA was then transcribed in vitro to produce antisense RNA in an amplification step using the T7 promoter-element (see, e.g., Luo et al. (1999) Nature Med 5:117-122). This antisense RNA was then converted into cDNA. The second set of cDNAs was again transcribed in vitro, using the T7 promoter, to further amplify the antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling. Fluorescent probes were generated by adding control RNA to the final round antisense RNA mix, and synthesizing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red).

The differential expression assay was performed by mixing equal amounts of probes from tumor cells and normal cells of the same patient. The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed. using BioDiscovery Autogene software, and the data from each scan set normalized to provide for a ratio of expression relative to normal. Data from the microarray experiments was analyzed according to the algorithms described in U.S. application serial No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level of fluorescence for each sequence on the array is expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

Table 1 summarizes the results of the P-cadherin differential expression analysis with tissues obtained from 33 colon cancer patients. Table 1 also contains a summary of pathological evaluations for these same patients. A polynucleotide is said to represent a significantly differentially expressed gene between two samples when there is detectable levels of expression in at least one sample and the ratio value is greater than at least about 1.2 fold, preferably greater than at least about 1.5 fold, more preferably greater than at least about 2.0 fold, where the ratio value is calculated using the method described above.

A differential expression ratio of 1 indicates that the expression level of the gene in the tumor cell was not statistically different from expression of that gene in normal colon cells of the same patient. A differential expression ratio significantly greater than 1 in cancerous colon cells relative to normal colon cells indicates that the gene is increased in expression in cancerous cells relative to normal cells, indicating that the gene plays a role in the development of the cancerous phenotype, and may be involved in promoting metastasis of the cell. Detection of gene products from such genes can provide an indicator that the cell is cancerous, and may provide a therapeutic and/or diagnostic target. It can be clearly seen from the results that P-cadherin is a significantly differentially exposed gene in colon cancer tissues of a majority of tested colon cancer patients.

### Example 4

### Differential Expression of P-Cadherin

Quantitative PCR of a colorectal carcinoma cell line, SW620, was used to analyze expression of P-cadherin. Quantitative real-time PCR was performed by first isolating RNA from cells using a Roche RNA Isolation kit according to manufacturer's directions. One microgram of RNA was used to synthesize a first-strand cDNA with MMLV reverse transcriptase (Ambion) in the manufacturers buffer and recommended concentrations of oligo dT, nucleotides, and Rnasin. This first-strand cDNA served as a template for quantitative real-time PCR using the Roche light-cycler as recommended in the machine manual. P-cadherin was amplified with the forward primer ACGTGCACCTTTCTCTGTCTGACCA (CADP1900)(SEQ ID NO:4) and reverse primer AAAAGCAGACCAGCAGGAGGAA (CADP2077)(SEQ ID NO:5). PCR product was quantified based on the cycle at which the amplification entered the linear phase of amplification in comparison to an internal standard and using the software supplied by the manufacturer. Small differences in amounts of total template in the first-strand cDNA reaction were eliminated by normalizing to amount of actin amplified in a separate quantitative PCR reaction using the forward primer 5′-CGGGAMTCGTGCGTGACATTMG-3′ (SEQ ID NO:6) and the reverse primer: 5′-TGATCTCCTTCTGCATCCTGTCGG-3′ (SEQ ID NO:7).

### Example 5

### Antisense Regulation of P-Cadherin Expression

Additional functional information on P-cadherin was generated using antisense knockout technology. P-cadherin expression in cancerous cells was further analyzed to confirm the role and function of the gene product in tumorgenesis, e.g., in promoting a metastatic phenotype.

A number of different oligonucleotides complementary to P-cadherin mRNA were designed as potential antisense oligonucleotides, and tested for their ability to suppress expression of P-cadherin. The ability of each designed antisense oligonucleotide to inhibit gene expression was tested through transfection into SW620 colon colorectal carcinoma cells. (It should be noted that this cell line, while being well suited for use in proliferation experiments, does not express high levels of P-cadherin mRNA).

For each transfection mixture, a carrier molecule, preferably a lipitoid or cholesteroid, was prepared to a working concentration of 0.5 mM in water, sonicated to yield a uniform solution, and filtered through a 0.45 μm PVDF membrane. The antisense or control oligonucleotide was then prepared to a working concentration of 100 μM in sterile Millipore water. The oligonucleotide was further diluted in OptiMEM™ (Gibco/BRL), in a microfuge tube, to 2 μM, or approximately 20 μg oligo/ml of OptiMEM™. In a separate microfuge tube, lipitoid or cholesteroid, typically in the amount of about 1.5-2 nmol lipitoid/μg antisense oligonucleotide, was diluted into the same volume of OptiMEM™ used to dilute the oligonucleotide. The diluted antisense oligonucleotide was immediately added to the diluted lipitoid and mixed by pipetting up and down. Oligonucleotide was added to the cells to a final concentration of 30 nM.

The level of target mRNA (P-cadherin) in the transfected cells was quantitated in the cancer cell lines using the Roche LightCycler™ real-time PCR machine. Values for the target mRNA were normalized versus an internal control (e.g., beta-actin). For each 20 μl reaction, extracted RNA (generally 0.2-1 μg total) was placed into a sterile 0.5 or 1.5 ml microcentrifuge tube, and water was added to a total volume of 12.5 μl. To each tube was added 7.5 μl of a buffer/enzyme mixture, prepared by mixing (in the order listed) 2.5 μl H₂O, 2.0 μl 10X reaction buffer, 10 μl oligo dT (20 μmol), 1.0 μl dNTP mix (10 mM each), 0.5 μl RNAsin® (20u) (Ambion, Inc., Hialeah, Fla.), and 0.5 μl MMLV reverse transcriptase (50u) (Ambion, Inc.). The contents were mixed by pipetting up and down, and the reaction mixture was incubated at 42° C. for 1 hour. The contents of each tube were centrifuged prior to amplification.

An amplification mixture was prepared by mixing in the following order: 1×PCR buffer 11, 3 mM MgCl₂, 140 μM each dNTP, 0.175 μmol each oligo, 1:50,000 dil of_SYBR® Green, 0.25 mg/ml BSA, 1 unit Taq polymerase, and H₂O to 20 μl. (PCR buffer 11 is available in 10×concentration from Perkin-Elmer, Norwalk, Conn.). In 1×concentration it contains 10 mM Tris pH 8.3 and 50 mM KCl. SYBR® Green (Molecular Probes, Eugene, Oreg.), a dye which fluoresces when bound to double stranded DNA. As double stranded PCR product is produced during amplification, the fluorescence from SYBRO Green increases. To each 20 μl aliquot of amplification mixture, 2 μl of template RT was added, and amplification was carried out according to standard protocols.

The following antisense oligonucleotides and their reverse controls were used in the transfection assays:

Cadherin P AS/RC sequences

| | | | | |
|---|---|---|---|---|
| cadh-p:P2807 | CHIR44-2807AS | AGGGTTGAGCATTTTACGGCAGGTG | 25 (7-11-7) | (SEQ ID NO. 8) |
| cadh-p:P2807RC | CHIR44-2807RC | ATGGACGGCATTTTACGAGTTGGGA | 25 (7-11-7) | (SEQ ID NO. 9) |
| cadh-p:P2258 | CHIR44-2258AS | CATGGGTGTCGGGATGATGGTTGGT | 25 (7-11-7) | (SEQ ID NO. 10) |
| cadh-p:2258RC | CHIR44-2258RC | TGGTTGGTAGTAGGGCTGTGGGTAC | 25 (7-11-7) | (SEQ ID NO. 11) |
| cadh-p:P0672 | CHIR44-0672AS | ACTTGGGCTTGTGGTCATTCTGGTC | 25 (7-11-7) | (SEQ ID NO. 12) |
| cadh-p:P0672RC | CHIR44-0672RC | CTGGTCTTACTGGTGTTCGGGTTCA | 25 (7-11-7) | (SEQ ID NO. 13) |
| cadh-p:P1556 | CHIR44-1556AS | CACAAACTGCTCATCCTCACGGTCG | 25 (7-11-7) | (SEQ ID NO. 14) |
| cadh-p:P1556RC | CHIR44-1556RC | GCTGGCACTCCTACTCGTCAAACAC | 25 (7-11-7) | (SEQ ID NO. 15) |
| cadh-p:P2446 | CHIR44-2446AS | AATCTTGGTCTTGGTCGGAGGCGG | 24 (7-10-7) | (SEQ ID NO. 16) |
| cadh-p:P2446RC | CHIR44-2446RC | GGCGGAGGCTGGTTCTGGTTCTAA | 24 (7-10-7) | (SEQ ID NO. 17) |

These antisense oligonucleotides were introduced into SW620 colon cancer cells. Additionally, a control was effected wherein the same amount of the reverse control oligonucleotides were introduced into SW620 cells.

The results of these experiments (not shown) were inconclusive. This is potentially explainable based on the relatively low levels of P-cadherin mRNA expressed by the SW620 cell line.

In particular, because the level of P-cadherin mRNA in SW620 cells is relatively low, when message quantitation experiments were conducted, the percentage knockout of antisense versus control could not be determined because the levels were too low (˜zero) to permit calculation of ratios.

By contrast (data summarized in Table 2), results with a different colon cancer cell line indicated (when the same P-cadherin antisense and reverse oligonucleotides were tested in other cells including KM12C cells), that knockout of P-cadherin expression was achieved. (It should be noted that the KM12C cell line, unfortunately, is not suitable for proliferation studies).

TABLE 2

| | Gene Message | | |
|---|---|---|---|
| Oligo | Levels | Actin 1 | Gene/Actin |
| Cadh-p 0672 AS | 1.482 | 0.652 | 2.2730 |
| Wild type RC | 20.53 | 1.16 | 17.6983 |
| Cadh-p 1556 AS | 0.733 | 0.26 | 2.8192 |
| Cadh-p 1556 RC | 4.281 | 0.355 | 12.0592 |
| Cadh-p 2258 AS | 0.335 | 0.712 | 0.4705 |
| Cadh-p 2258 RC | 1.927 | 0.443 | 4.3499 |
| Cadh-p 2446 AS | 0.583 | 1.152 | 0.5061 |
| Cadh-p 2446 RC | 14.22 | 0.692 | 20.5491 |
| Cadh-p 2807 AS | 0.286 | 0.891 | 0.3210 |
| Cadh-p 2807 RC | 7.052 | 0.559 | 12.6154 |
| Wild type | 18.53 | 1.345 | 13.7770 |

Additional proliferation data with A431 cells transfected with P-cadherin antisense oligos and reverse controls is contained in Tables 4 and 5. It can be seen from the results that proliferation was inhibited by antisense treatment.

TABLE 3

| | | Averages | | | | |
|---|---|---|---|---|---|---|
| A431 roliferation | | Day 0 | Day 1 | Day 2 | Day 3 | Day 4 |
| Non-transfected | Untransfected | 0.218 | 0.295 | 0.800 | 1.593 | 2.305 |
| | Control1 | | | | | |
| Non-transfected | Untransfected | 0.194 | 0.294 | 0.803 | 1.615 | 2.424 |
| | Control2 | | | | | |
| (+) Control | CHIR79-9 | 0.214 | 0.259 | 0.321 | 0.449 | 0.536 |
| (−) Control | CHIR79-9RC | 0.197 | 0.288 | 0.547 | 0.859 | 1.457 |
| (+) Control | CHIR120-11 | 0.218 | 0.220 | 0.100 | 0.095 | 0.106 |
| (−) Control | CHIR120-11RC | 0.201 | 0.279 | 0.432 | 0.646 | 1.125 |
| Antisense | CHIR44-1556 | 0.215 | 0.287 | 0.344 | 0.493 | 0.786 |
| Reverse Control | CHIR44-1556RC | 0.197 | 0.270 | 0.413 | 0.668 | 1.177 |
| Antisense | CHIR44-2258 | 0.213 | 0.286 | 0.490 | 0.822 | 1.361 |
| Reverse Control | CHIR44-2258RC | 0.203 | 0.296 | 0.596 | 1.005 | 1.819 |
| Antisense | CHIR44-2446 | 0.213 | 0.288 | 0.449 | 0.674 | 0.987 |
| Reverse Control | CHIR44-2446RC | 0.196 | 0.298 | 0.545 | 0.832 | 1.488 |

TABLE 4

| Standard Deviations | | | | | P-Value of T-Test | | | | |
|---|---|---|---|---|---|---|---|---|---|
| Day 0 | Day 1 | Day 2 | Day 3 | Day 4 | Day 0 | Day 1 | Day 2 | Day 3 | Day 4 |
| 0.007 | 0.019 | 0.020 | 0.032 | 0.019 | 0.0854 | 0.9561 | 0.8380 | 0.9638 | 0.245 |
| 0.017 | 0.007 | 0.006 | 0.001 | 0.150 | | | | | |
| 0.006 | 0.007 | 0.020 | 0.037 | 0.052 | 0.1037 | 0.0220 | 0.0004 | 0.0002 | 0.000 |
| 0.001 | 0.012 | 0.028 | 0.037 | 0.031 | | | | | |
| 0.006 | 0.008 | 0.002 | 0.017 | 0.028 | 0.3083 | 0.0006 | 0.0000 | 0.0000 | 0.000 |
| 0.024 | 0.006 | 0.005 | 0.032 | 0.077 | | | | | |
| 0.012 | 0.006 | 0.049 | 0.029 | 0.028 | 0.2779 | 0.2681 | 0.0976 | 0.0005 | 0.000 |
| 0.023 | 0.001 | 0.026 | 0.006 | 0.043 | | | | | |
| 0.004 | 0.005 | 0.017 | 0.028 | 0.024 | 0.4857 | 0.0836 | 0.0005 | 0.0011 | 0.000 |
| 0.022 | 0.005 | 0.006 | 0.026 | 0.073 | | | | | |
| 0.011 | 0.012 | 0.020 | 0.049 | 0.055 | 0.3076 | 0.2579 | 0.0054 | 0.0141 | 0.000 |
| 0.022 | 0.004 | 0.023 | 0.043 | 0.034 | | | | | |

### Example 6

### Effect of P-Cadherin Expression on Proliferation

The effect of P-cadherin on cell proliferation was assessed in SW620 colon colorectal carcinoma cells. As noted previously, this cell line expresses low levels of P-cadherin. Transfection was carried out as described above in Example 5.

Cells were plated to approximately 60-80% confluency in 96-well dishes. Antisense or reverse control oligonucleotide was diluted to 2 μM in OptiMEM™ and added to OptiMEM™ into which the delivery vehicle, lipitoid 116-6 in the case of SW620 cells, had been diluted. The oligo/delivery vehicle mixture was then further diluted into medium with serum on the cells. The final concentration of oligonucleotide for all experiments was 300 nM, and the final ratio of oligo to delivery vehicle for all experiments was 1.5 nmol lipitoid/μg oligonucleotide. Cells were transfected overnight at 37° C. and the transfection mixture was replaced with fresh medium the next morning.

Transfection of both antisense oligonucleotides into SW620 colorectal carcinoma cells resulted in a decreased rate of proliferation compared to matched reverse control (RC) oligonucleotides.

### Example 7

### In vitro Assay Using Anti-P-Cadherin Antibody to Disrupt Cell-Cell Contact

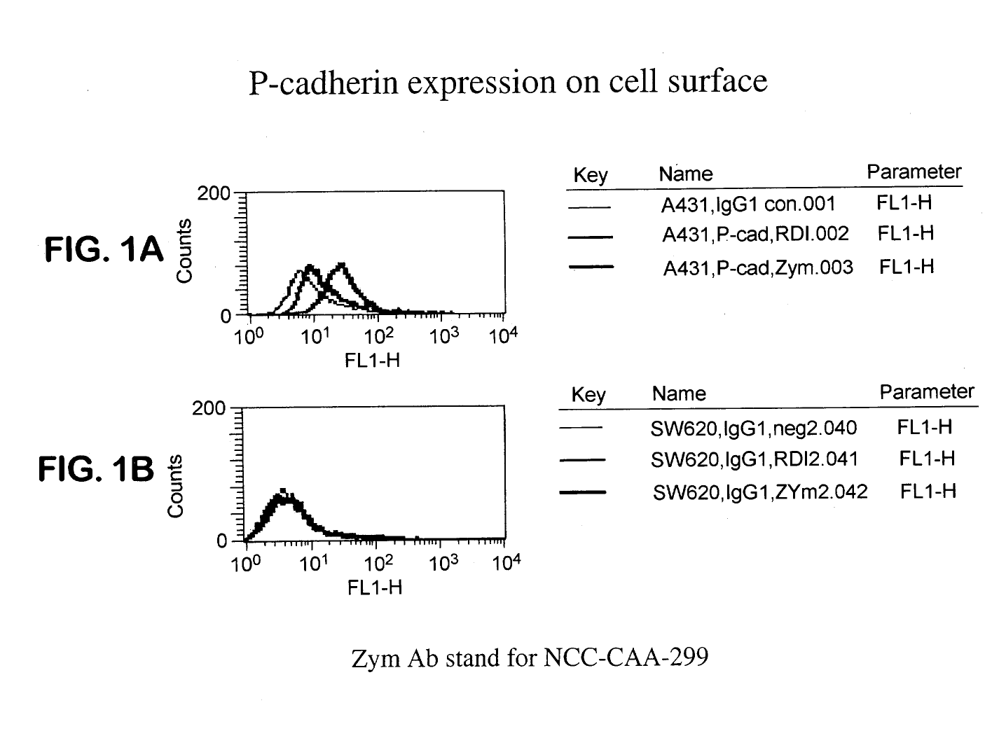

An in vitro experiment was conducted to test the potential of an anti-P-cadherin antibody for cancer immunotherapy. Particularly, an epithelial tumor cell line A-431 which expresses a moderate amount of P-cadherin was used as a model system to evaluate the therapeutic potential of two anti-P-cadherin antibodies (cat #NCC-CAD-299, Zymed and clone#RDI-PCADHER abm obtained from RDI). A control mouse IgG1 was also used in this experiment (see FIG. 1). The antibodies were respectively added to cells contained in 96 cell culture plates at the time of plating, the cultures which were then incubated for four days. Cell growth patterns were observed by light microscope and proliferation was measured by fluorescence staining of DNA (Quantos Kit).

The experiment showed that the anti-P-cadherin antibody, NCC-CAD-299, disrupted cell-cell contact in A431 cells. By contrast, P-cadherin expressing A431 cells grew in tightly associated clusters when no antibody was added or in the presence of the control irrelevant antibody. The cells in the presence of the anti-P-cadherin antibody (NCC-CAD-299) grew in a scattered pattern. These results are shown in FIG. 2. The disparate results observed with the two antibodies are hypothesized to be attributable to differences between the two antibodies.

### Example 8

### Effect of Anti-P-Cadherin Antibody on Cell Proliferation

An experiment was also conducted to evaluate the effect of the same anti-P-cadherin antibodies, NCC-CAD-299 and anti-P-cadherin antibody RDI-PCADHER abm (obtained from RDI) on cell proliferation. A-431 cells were plated in 96 cell plates with anti-P-cadherin and cell proliferation measured at day four. Cell proliferation of the control cultures were also evaluated in the presence of the same amount of an irrelevant isotype matched IgG1 antibody.

The results obtained (in FIG. 12) show that A-431 growth was inhibited in the presence of the NCC-CAD-299 antibody, but not in the presence of the IgG1 isotype control antibody, or the other anti-P-cadherin antibody obtained from RDI. This inhibition was comparable to that seen with a positive control antibody 225 (anti-EGFR antibody). These results are contained in FIG. 12.

These results suggest that while P-cadherin is known as an adhesion molecule and other researchers have observed that its expression correlates to poor survival, that P-cadherin may play a dual role in modulating cell-cell contact and cell proliferation. Consequently, disrupting or blocking P-cadherin should inhibit tumor growth and/or migration. This may also have an inhibitory role on metastasis as the antibody may inhibit migration and/or attachment of tumor cells to other sites.

### Example 9

### Expression, and Purification of Cadherins for Immunization and Screening:

The expression constructs for all Cadherin proteins were obtained by RT-PCR using the Gateway™ system (GIBCO-BRL). Total RNA from KM12-L4 cells for P-Cadherin, total human RNA from colon for E-Cadherin, total human RNA from the heart for H-Cadherin and total human brain RNA for N-Cadherin constructs were used as templates for the cDNA synthesis. The products of the PCR reactions were recombined into the vector pDONR201 according to the Gateway procedures. The correct clones were selected after sequence verification.

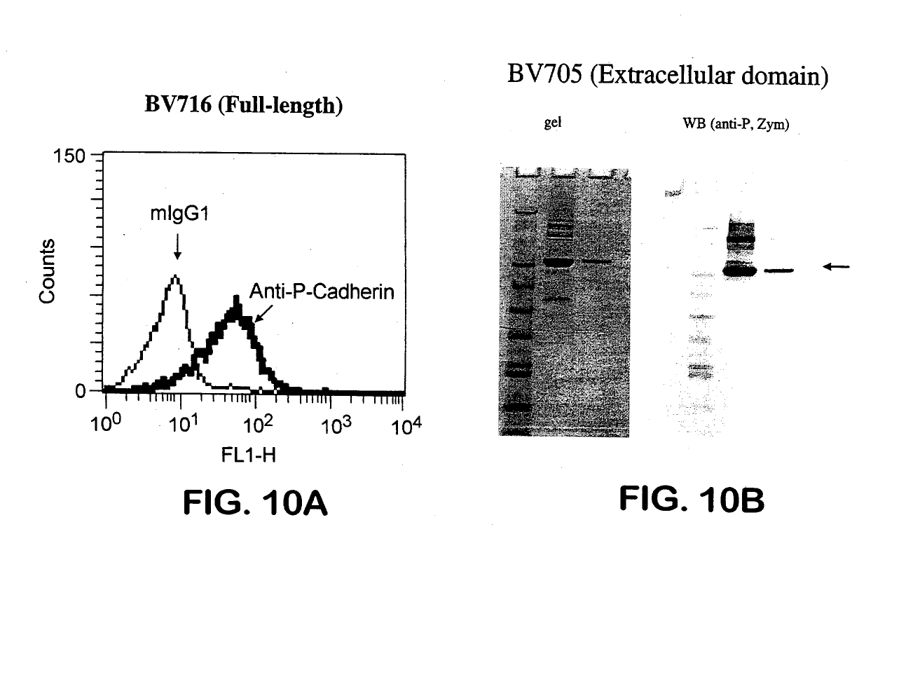

The sequences of cDNA corresponding to different forms of human P-Cadherin were digested and inserted into a baculovirus vector pMelBac or pBlueBac, depending on the requirements. The plasmids were co-transfected with a wild-type viral DNA into insect cells (Kitts et al., Nucleic Acids Res. 18:5667-5672 (1990)). The recombinant baculovirus was isolated by plaque purification. Western blot analysis with anti-His antibody and anti-P-Cadherin antibody was performed to confirm the expression of the proteins. For cadherin productions, TN5 cells were infected with the appropriate baculovirus at multiplicity of infection (moi) 2-10 in protein-free medium. The BV716-infected TN5 cells were harvested at 48 hours post infection to analyze the cell-bound P-Cadherin (FIG. 10A). Each soluble cadherin was collected separately from the culture media, and purification was performed. Purification of the soluble cadherins were conducted on a Q SepharoseFF column. The production of the Cadherin was measured by SDS-PAGE and Western Blotting (FIG. 10B). The sequence of N terminus of the protein has been confirmed by Edmunds degradation. Endotoxin levels in the final product are below detection for all of the cadherins. The Cadherin vectors and recombinant baculovirus are summarized in Table 3.

To get an EC1 domain of P-cadherin, the PCAD-EC1/Sag fusion was generated by PCR using the mammalian expression vector HBSag-EC1 (S. Schleyer) as the template DNA. The promoter/fusion gene fragment was cloned into the yeast expression vector pBS24.1. The resultant plasmid was named PCAD-EC1/Sag. The PCAD-EC1/Sag plasmid was transformed into yeast AD3 and plated on selective medium. The single colon was selected and cultured for production of P-Cadherin EC1 protein. The protein from the lysed yeast cells was used to determine the binding sites of in-house P-Cadherin antibodies by Western Blot.

### Example 10

### Immunization and Cell Fusion

In order to develop a therapeutic P-Cadherin antibody, six- to ten-week-old transgenic mice are obtained. The immunizations were performed via intraperitoneal (IP), footpad (FP), or base of tail (BOT) plus IP, with P-Cadherin-expressing insect cells BV716 and/or soluble proteins BV703. In IP and BOT immunization, the antigens were emulsified with complete Freunds adjuvant (CFA) for priming immunization and incomplete Freunds adjuvant (IFA) for booster immunization. Total immunizations are performed 5 times at 2-week intervals. In the mice immunized via FP, the soluble P-Cadherin emulsified with TiterMax adjuvant for priming immunization, and Alum adjuvant for booster immunization, performed 8 times at 2-day intervals.

The animals are bled and the P-Cadherin antisera were tested by ELISA. The titers in the immunized mice are determined and compared. Thereafter, fusions are performed either by polyethylene glycol (PEG) for the mice immunized via IP, or electroporation for the mice immunized via FP and BOT plus IP.

### Example 11

### Antibody Characterization

The fused cells were screened with soluble P-cadherin BV703 by ELISA, and further screened on P-Cadherin-expressing tumor cells, A431 by FACS. The cell-surface-binding hybridomas are cloned by limiting dilution. The cross-reactions to other P-Cadherin family members is also tested by ELISA against soluble E-Cadherin (BV744), N-Cadherin (BV751), and H-Cadherin (BV767), as well as by FACS against E-Cadherin-expressing cells (MCF-7) and H-Cadherin-expressing cells (H460).

Using these methods, clones are obtained that demonstrate P-Cadherin cell surface domain-binding. Three antibodies are screened to evaluate their ability to inhibit cell-cell contacts. The biological functions of antibodies that bind the EC1 domain are evaluated.

### Example 12

### Migration Assay

The effect of P-cadherin-specific antibodies on tumor cell (HCT116, a colon cancer cell lone) adhesion and invasion will be tested by a migration assay. FALCON HTS LuoroBlok Inserts from Becton Dickinson will be used in this assay. HCT 116 cells will be incubated with anti-P-cadherin antibody or control antibodies for 30 min, and the 8×10e4 cells in 200 ul medium will be loaded into an 8 um pore size insert. Six hundreds of medium will be added to the bottom chambers, supplemented with 1% BSA as a negative control, or appropriate chemoattractants (EGF or fibronectin) as a positive control. The cells will be allowed to migrate for 22 hours in 37 C, 5% CO₂. The migrated cell swill be stained with fluorescence dye and measured by fluorescence reader.

### Example 13

### Intracelullar Adhesion Assay

P-cadherin antibodies also have application in intracellular adhesion assays. For example, dermal fibroblast or HUVEC cells will be cultured to form a monolayer. P-cadherin-expressing tumor cells will be pre-labeled with the red fluorescent dye for 2 h, washed with HBSS, and harvested by treatment with 0.2% trypsin in HBSS containing 2 mM calcium for 30 min at 37° C. The cells will be incubated with anti-P-cadherin mAb (40 ug/ml) or control antibodies at 4° C. for 30 min, and then washed with HBSS. About 5000 cells will be added to dermal fibroblast or HUVEC monolayers in gelatin-coated, eight-well chamber slides and allowed to adhere for 30 min. After removal of non-adherent cells, slides will be fixed. The number of adherent cells per high-power field in triplicate wells will be counted under a fluorescence microscope (Volberg T., Geiger B., Kartenbeck J., Franke W. W., J. Cell Biol, 102:1832-1842 (1986)).

### Example 14

### Proliferation Assay

P-cadherin antibodies also have application in proliferation assays. For example, the direct inhibition of P-cadherin-expressing cells with the Ab will be checked by proliferation assay. The 1500-2000 A431 or HCT116 cells in 200 ul medium will be plated in 96-well plates, and incubated for 4 days with or without P-cadherein-specific antibodies. At day 4, the supernatants will be removed by dumping and the cells will be put into −80C at least 2 hours. The cells will be then thawed and Cell Proliferation Cyquant kit will be used to measure the proliferation rate, according to manufacture's instruction.

### Example 15

### ADCC/CDC Property Test

Antibodies having effector function may be superior for therapeutic use. For example, human IgG1 antibodies can be applied to treat cancer through an antibody-dependent cell-mediated cytotoxicity (ADCC) by binding to its Fc receptors, and a complement-dependent cytotoxicity (CDC) by fixing complement. LDH cytotoxicity Detection Kit will be used in both assays. In ADCC test, the P-cadherin-expressing cell lines A431 and HCT116 will be used as target cells, human peripheral blood mononuclear cells (PBMC) or natural killers (NK) as effector. Five thousands target cells per well will be plated in U-bottomed plate at effector to target ratio 100:1, 50:1 and 25:1 for PBMC, and 10:1, 5:1, and 2.5:1 for NK. The cells will be co-incubated for 4 hours and the supernatants will be collected to measure lactate dehydrogenase (LDH) activity with LDH assay kit. The CDC property will be tested in the same way as ADCC, except for adding human complement instead of human PBMC or NK.

### Example 16

### Anchorage-Independent Cell Viability Assay

P-cadherin antibodies also have application in anchorage-dependent cell assays. For example, in such an assay the cells are cultured in 1% agarose-coated dishes with or without antibody treatment. Viability was determined in triplicate samples by trypan blue exclusion assay, and the survival index was calculated as: Survival index=Number of live cells/Total number cells.

### Example 17

### AKT/PKB Signaling Check

P-cadherin potentially is involved in promoting tumor cell survival by activating antiapoptotic protein and subsequently stabilizes B-catenin and inactivates proapoptoic factor Bad. To determine whether anti-P-cadherin antibody affects Akt/PKB activation, the confluent cells will be serum starved overnight and treated with EGTA (final concentration, 4 mM) for 30 min to disrupt Ca2+dependent, P-cadherin-mediated adhesion. The medium will be then replaced with serum-free medium containing 2 mM calcium and incubated for 10 min with P-cadherin-specific blocking antibody or control antibody at 40 ug/ml before calcium restoration. At 30 min after calcium restoration, cells will be lysed and immunoblotted with antibodies against Akt/PKB and phospho-Akt/PKB (Ser473) (Gang Li, Kapaettu Satyamoorthy and Meenhard Heryin, Intracelluceullar Interactions Promote Survival and Migration of Melanoma Cells. Cancer Research 61:3819-3825 (2001)).

### Example 18

### Cell Cycle Checkpoint Assay

The P-cadherin-expressing tumor cells will be plated into 6-well plate and incubated with anti-P-cadherin antibody for 24 hours. The cells will be then harvested, and washed with PBS containing sodium azide. Cell cycle distribution and apoptotic DNA profiles of cells will be determined by propidium iodide (PI) staining in the presence of RnaseA and analyze by flow cytometry.

### Example 19

### In vivo Evaluation

As discussed upon, the efficacy of P-cadherin antibodies can be confined in in vivo assays. The P-cadherin-specific antibodies will be tested in vivo for inhibition of P-cadherin-expressing tumor. A colon cancer cell line, HCT116, and an epidermoid cell line, KM12 will be used in xenograft tumors in nude mice. The tumor-bearing mice will be treated with P-Cadherin-specific antibody is administered via ip injection. The tumor volumes and the animal survivals will be monitored accordingly.

TABLE 5

Summary of constructs for P-cadherin expression

| | Plasmid | Baculovirus | | | |
|---|---|---|---|---|---|
| Gene | name | name | Description | Primers | Sequence |
| P-Cadherin | Orf94-17 | BV716 | Cell-bound, Full-length | 5′ | TTCTTCGGATCCGATTGGGTGGTTGCTCCAATA |
| | | | w/o signal peptide | | TCTGTCCCT |
| | | | | 3′ | GTGTATGAATTCCTAGTCGTCCTCCCCGCCACC |
| | | | | | GTACATGTC |
| P-Cadherin | Orf103 | BV703 | Soluble, EC, w/o signal | 5′ | GGGACAAGTTTGTACAAAAAAGCAGGCTCAGAT |
| | | | peptide, C-ter His Tag, | | TGGGTGGTTGCTCCAATATCTGTCCCTGAA |
| | | | | 3′ | GGGACCACTTTGTACAAGAAAGCTGGGTCATCT |
| | | | | | AGAGTGATGGTGATGGTGATGTTTCCAGGGTCC |
| | | | | | AGGGCAGGTTTCGACATG |
| P-Cadherin-EC1 | | Yeast, PCAD-EC1/SAg | Soluble, EC1 | 5′ | CTCTGACCATGGGACAGTGGAACTCCCGAAGAC |
| EC1 | | | | | ACAAG |
| | | | | 3′ | CTCTGAGTCGACTTAAATGTATACCCAGAGAC |
| P-Cadherin | Orf104 | BV705 | Soluble, EC, w/signal | 5′ | GGGACAAGTTTGTACAAAMAGCAGGCTCGTGC |
| | | | peptids, W/His Tag | | CGGGCGGTCTTCAGGGAGGCTGAAG |
| N-Cadherin | Orf121 | BV751 | Soluble, EC, w/o signal | 5′ | GGGACAAGTTTGTACAAAAAAGCAGGCTCAATG |
| | | | peptide, w/C-ter His | | GACTGGGTCATCCCTCCAATCAACTTGCCA |
| | | | Tag | | |
| | | | | 3′ | GGGACCACTTTGTACAAGAAAGCTGGGTTCATC |
| | | | | | TAGAGTGATGGTGATGGTGATGCCTGTCCACAT |
| | | | | | CTGTGCAGTCCCCGTTGGAGTC |
| H-Cadherin | Orf114 | BV767 | Soluble, EC, w/signal | 5′ | GGGACAAGTTTGTACAAAAAAGCAGGCTCAGAA |
| | | | peptide | | GATTTGGACTGCACTCCTGGATTTCAG |
| | | | w/C-ter His Tag, | | |
| | | | | 3′ | GGGACCACTTTGTACAAGAAAGCTGGGTTCATC |
| | | | | | TAGAGTGATGGTGATGGTGATGCGCCGCGTTG |
| | | | | | CAGTCCACTTTGGAATTCCTGCA |
| E-Cadherin | Orf119 | BV744 | Soluble, EC, w/o signal | 5′ | GGGACAAGTTTGTACAAAkAAGCAGGCTCAATG |
| | | | peptide | | GACTGGGTTATTCCTCCCATCAGCTGCCCAGAA |
| | | | w/C-ter His Tag | | |
| | | | | 3′ | GGGACCACTTTGTACAAGAAAGCTGGGTTCATC |
| | | | | | TAGAGTGATGGTGATGGTGATGCGCCGCGTTG |
| | | | | | CAGTCCACTTTGGMTTCCTGCA |

EC: extracellular domains

What is claimed:

1. A method of treating a cancer characterized by the overexpression and/or upregulation of P-cadherin comprising the administration of an effective amount of at least one P-cadherin antagonist, optionally conjugated to a therapeutic agent.

2. A method of inhibiting the migration, adhesion and/or proliferation of a P-cadherin expressing cancer comprising administering a subject in need of such treatment an effective amount of a P-cadherin antagonist, optionally conjugated to a therapeutic agent.

3. A method of treating or preventing a digestive cancer characterized by the overexpression and/or upregulation of P-cadherin comprising the administration of an effective amount of at least one P-cadherin antagonist, optionally conjugated to a therapeutic agent.

4. A method of inhibiting the migration, adhesion and/or proliferation of digestive cancer cells that express P-cadherin comprising administering an effective amount of a P-cadherin antagonist, optionally conjugated to a therapeutic agent.

5. A method of treating a colon or colorectal cancer characterized by the overexpression and/or upregulation of P-cadherin comprising the administration of an effective amount of at least one P-cadherin antagonist, optionally conjugated to a therapeutic agent.

6. A method of inhibiting the migration, adhesion and/or proliferation of colon cancer cells in a subject in need of such treatment comprising administering an effective amount of a P-cadherin antagonist, optionally conjugated to a therapeutic agent.

7. A method of treating or preventing a cancer characterized by the overexpression and/or upregulation of P-cadherin comprising the administration of an effective amount of at least one P-cadherin antagonist or P-cadherin-binding antibody fragment, optionally conjugated to a therapeutic agent.

8. A method of treating or preventing a cancer characterized by the overexpression and/or upregulation of P-cadherin comprising the administration of an effective amount of a ribozyme or antisense oligonucleotide that modulates P-cadherin expression, optionally conjugated to a therapeutic agent.

9. A method of treating or preventing a digestive cancer characterized by the overexpression and/or upregulation of P-cadherin comprising the administration of an effective amount of at least one anti-P-cadherin antibody or P-cadherin-binding antibody fragment, optionally conjugated to a therapeutic agent.

10. A method of treating or preventing a digestive cancer characterized by the overexpression and/or upregulation of P-cadherin comprising the administration of an effective amount of at least one ribozyme or antisense oligonucleotide that modulates P-cadherin expression, optionally conjugated to a therapeutic agent.

11. A method of treating or preventing a colon or colorectal cancer characterized by the overexpression and/or upregulation of P-cadherin comprising the administration of an effective amount of an anti-P-cadherin antibody or P-cadherin-binding antibody fragment, optionally conjugated to a therapeutic agent.

12. The method of any one of claims 1-11 where the P-cadherin antagonist is a monoclonal antibody.

13. The method of claim 12 wherein said antibody is a humanized antibody.

14. The method of claim 12 wherein said antibody is a chimeric antibody.

15. The method of claim 12 wherein said antibody is a human antibody.

16. The method of claim 12 wherein said antibody is a single chain antibody.

17. The method of claim 12 wherein said antibody comprises human IgG1, IgG2, IgG3 or IgG4 constant domains.

18. The method of claim 12 wherein said antibody possesses ADCC and/or CDC activity.

19. The method of claim 12 wherein said antibody induces apoptosis.

20. A human, chimeric or humanized anti-P-cadherin antibody which is suitable for treatment of a cancer characterized by P-cadherin overexpression and/or upregulation, optionally conjugated to a therapeutic agent.

21. The composition of claim 20 wherein said antibody is a humanized antibody.

22. The composition of claim 20 wherein said antibody is a chimeric antibody.

23. The composition of claim 20 wherein said antibody is a human antibody.

24. The composition of claim 20 wherein said antibody is a single chain antibody.

25. The composition of claim 20 wherein said antibody comprises human IgG1, IgG2, IgG3 or IgG4 constant domains.

26. The composition of claim 20 wherein said antibody possesses ADCC and/or CDC activity.

27. The composition of claim 20 wherein said antibody induces apoptosis.

28. A transgenic non-human animal that expresses a recombinant antibody that specifically binds P-cadherin.

29. A transgenic animal that expresses a recombinant human antibody that specifically binds P-cadherin.

30. The animal or transgenic of claim 28 wherein said antibody is an IgG1.

31. A ribozyme that modulates P-cadherin expression in a P-cadherin expressing cell.

32. An antisense oligonucleotide that modulates P-cadherin expression in a P-cadherin expressing cell.

33. The animal of claim 28 wherein said antibody is a humanized antibody.

34. The animal of claim 28 wherein said antibody is a chimeric antibody.

35. The animal of claim 28 wherein said antibody is a human antibody.

36. The animal of claim 28 wherein said antibody is a single chain antibody.

37. The animal of claim 28 wherein said antibody comprises human IgG1, IgG2, IgG3 or IgG4 constant domains.

38. The animal of claim 28 wherein said antibody possesses ADCC and/or CDC activity.

39. The animal of claim 28 wherein said antibody induces apoptosis.

40. A pharmaceutical composition adopted for the treatment of a cancer characterized by the overexpression and/or upregulation of P-cadherin that comprises a pharmaceutically effective amount of at least one human, chimeric or humanized antibody or antibody fragment that specifically binds P-cadherin and a pharmaceutically acceptable carrier.

41. A pharmaceutical composition adopted for the treatment of a cancer characterized by the overexpression and/or upregulation of P-cadherin comprising a pharmaceutically effective amount of at least one ribozyme or antisense oligonucleotide that modulates P-cadherin expression and a pharmaceutically acceptable carrier.

42. A recombinant host cell that expresses a human, humanized or chimeric antibody or antibody fragment that specifically binds P-cadherin.

43. The host cell of claim 42 which is an insect, mammalian or yeast cell.

44. A method of determining the presence of a cancer involving the overexpression and/or upregulation of P-cadherin comprising:
(i) obtaining a cell sample from a patient to be diagnosed for the presence or absence of a cancer involving the overexpression and/or upregulation of P-cadherin;
(ii) determining the level of expression of P-cadherin in said cell sample;
(iii) comparing said levels of P-cadherin expression to a normal cell sample; and
(iv) correlating said level of P-cadherin expression in said patient cell sample relative to the normal cell sample to a positive or negative diagnosis of a cancer associated with the overexpression and/or upregulation of P-cadherin.

45. A method of inhibiting the proliferation of colon cancer in a subject in need of such treatment comprising administering an effective amount of an antibody to P-cadherin or a fragment thereof that specifically binds P-cadherin.

46. A method of inhibiting at least one of the adhesion, proliferation and/or migration of a cancer cell associated with upregulation of P-cadherin comprising administering an inhibitory effective amount of at least one P-cadherin antagonist, optionally conjugated to a therapeutic agent.

47. A method of inhibiting at least one of the adhesion, migration and/or proliferation of colon cancer cells characterized by upregulation of P-cadherin comprising administering to a subject in need of such treatment an inhibitory effective amount of at least one P-cadherin antibody, optionally conjugated to a therapeutic agent.

48. A method of inhibiting at least one of the adhesion, migration and/or proliferation of a cancer cell associated with upregulation of P-cadherin comprising administering an inhibitory effective amount of at least one P-cadherin antibody, optionally conjugated to a therapeutic agent.

49. A method of inhibiting at least one of the adhesion, migration and/or proliferation of colon cancer cells characterized by upregulation of P-cadherin comprising administering to a subject in need of such treatment an inhibitory effective amount of at least one antibody that specifically binds P-cadherin, optionally conjugated to a therapeutic agent.

50. A method of therapy comprising preferentially targeting P-cadherin overexpressing tumor tissues by administration of a P-cadherin binding antibody or antibody fragment.

51. The method of any one of claims 44-49 wherein said antibody is a human antibody.

52. The method of any one of claims 44-49 wherein said antibody is a chimeric antibody

53. The method of any one of claims 44-49 wherein said antibody is a humanized antibody.

54. The method of any one of claims 44-49 wherein said antibody is attached to a chemotherapeutic agent.

55. The method of anyone of claims 44-49 wherein said antibody is attached to a radionuclide.

56. The method of any one of claims 4449 wherein said antibody is attached to a toxin.

57. A method of screening for an anti-P-cadherin antibody having potential therapeutic activity comprising screening a population of anti-P-cadherin antibodies for those that inhibit proliferation of tumor cells.

58. A method of screening for an anti-P-cadherin antibody having potential therapeutic activity comprising screening a population of anti-P-cadherin antibodies for those that induce apoptosis of tumor cells.

59. A method of screening for an anti-P-cadherin antibody having potential therapeutic activity comprising screening a population of anti-P-cadherin antibodies for those that posses ADCC and/or CDC activity.

60. A method of screening for an anti-P-cadherin antibody having potential therapeutic activity comprising screening a population of anti-P-cadherin antibodies for those that inhibit tumor cell migration

61. A method of screening for an anti-P-cadherin antibody having potential therapeutic activity comprising screening a population of anti-P-cadherin antibodies for those that inhibit metastasis.

62. A method for screening for an anti-P-cadherin antibody that binds to the EC1 domain comprising screening a population of anti-P-cadherin antibodies for those that bind the EC1 domain.

63. A method for screening for an anti-P-cadherin antibody that possess one of the following properties by screening population of anti-P-cadherin antibody population for an antibody that possesses at least one of the properties:
(i) interferes with P-cadherin strand formation;
(ii) interferes with cis dimer formation of P-cadherin proteins;
(iii) blocks or inhibits calcium binding by P-cadherin; and
(iv) interferes with P-cadherin domain alignment.

64. An anti-P-cadherin monoclonal antibody produced by the method of any one of claims 58-63.
